(19) United States
(12) Patent Application Publication
(10) Pub. No.: US 2021/0380547 A1
(43) Pub. Date: Dec. 9, 2021

(54) UREA DERIVATIVES FOR TREATING AND/OR PREVENTING CANCER

(71) Applicants: CENTRE NATIONAL DE LA RECHERCHE SCIENTIFIQUE, PARIS (FR); INSTITUT NATIONAL DE LA SANTE ET DE LA RECHERCHE MEDICALE, PARIS (FR); UNIVERSITE DE PARIS, PARIS (FR); UNIVERSITE CÔTE D'AZUR, NICE (FR)

(72) Inventors: RACHID BENHIDA, NICE (FR); GILLES PAGES, MONACO (MC); MAEVA DUFIES, NICE (FR); LUC DEMANGE, MENTON (FR); CYRIL RONCO, NICE (FR); OLEKSANDR GRYTSAI, NICE (FR)

(21) Appl. No.: 17/285,495

(22) PCT Filed: Oct. 17, 2019
(86) PCT No.: PCT/EP2019/078274

§ 371 Date: Apr. 15, 2021

(30) Foreign Application Priority Data
Oct. 17, 2018 (EP) 18306362.7

(51) Int. Cl. C07D 277/82; A61P 35/00; C07D 235/30

(52) U.S. Cl. CPC C07D 277/82; C07D 235/30; A61P 35/00

(57) ABSTRACT

The present invention relates to a compound or a pharmaceutically acceptable salt thereof of formulae (I) and (II), and a pharmaceutical composition comprising such compound for use for treating a cancer, particularly a cancer overexpressing CXCR1 and CXCR2 receptors, such as medulloblastoma, head and neck and kidney cancer. The invention further relates to such compounds for use for treating macular degeneration.

### FIELD OF THE INVENTION

The present invention relates to the field of medicine, in particular to the use of CXCR1 and CXCR2 receptors antagonists in the treatment of cancer and disorders characterized by undesirable excessive angiogenesis, such as macular degeneration.

### BACKGROUND OF THE INVENTION

Angiogenesis is a process comprising the formation of new capillary blood vessels from pre-existing microvessels. Angiogenesis normally occurs during embryonic development, tissue regeneration, wound healing, and corpus luteum development that is a cyclical change in the female reproductive system.

However, it exists a large number of diseases induced by dysregulated angiogenesis. Such diseases associated with angiogenesis occurring in pathological conditions include hemangioma, angiofibroma, vascular malformation and cardiovascular diseases, such as arteriosclerosis, vascular adhesion, and scleroderma. Ocular diseases associated with angiogenesis include corneal graft angiogenesis, neovascular glaucoma, diabetic retinopathy, corneal diseases induced by new blood vessels, macular degeneration, pterygium, retinal degeneration, retrolental fibroplasia, granular conjunctivitis, and the like. Furthermore, angiogenesis-related diseases may include chronic inflammatory diseases such as arthritis, cutaneous diseases such as psoriasis, capillarectasia, pyogenic granuloma, seborrheic dermatitis, acne, Alzheimer's disease, and obesity.

Among these disorders, macular degeneration, such as age-related macular degeneration, impacts millions of older adults every year. The disease affects central vision and can sometimes make it difficult to read, drive or perform other activities requiring fine, detailed vision. When the macula is damaged, the eye loses its ability to see detail, such as small print, facial features or small objects. The damaged parts of the macula often cause scotomas or localized areas of vision loss. Known treatments for macular degeneration or age-related macular degeneration include anti-VEGF (Vascular Endothelial Growth Factor) agents that cause regression of abnormal blood vessel growth and improvement of vision when injected directly into the vitreous humour of the eye. Examples of these agents include the monoclonal antibodies to VEGF, ranibizumab (marketed as “Lucentis”), bevacizumab (marketed as “Avastin”) and pegatanib (marketed as “Macugen”). Treatments involving the use of these drugs are often expensive and often not efficient. There is, therefore, a clear need to identify alternative beneficial cellular targets for the treatment macular degeneration and develop suitable therapies around these targets.

Angiogenesis plays also an important role in the growth and metastasis of cancer cells. Tumor is supplied with nutrition and oxygen necessary for growth and proliferation through new blood vessels, and the new blood vessels infiltrating into the tumor allow the cancer cells being metastasized to enter the blood circulation system and thus support metastasis of the cancer cells. Several therapeutic agents targeting VEGF-A₁₆₅, the main pro-angiogenic factor and its associated receptors, have been approved for cancer treatment. For instance, Bevacizumab (a recombinant humanized monoclonal antibody) and Sunitinib (small-sized kinase inhibitor targeting specific VEGF receptors) are commercially available under trademarks Avastin® and Sutent®, respectively. For the patients, these conventional drugs lead to an indisputable initial period of clinical benefit. However, they fail to definitively cure cancers. The treated primary tumors often relapse and remaining malignant cells disseminate to distant healthy tissues, inducing thereby metastases.

In cancer, inflammation and angiogenesis are two closely integrated processes. Indeed, the specific family of cytokines, the CXCL family, induces pro-angiogenic or anti-angiogenic signals depending on the presence or absence of the amino-acid triplet ELR (glycine-leucine-arginine) in their sequence. The pro-angiogenic ELR⁺CXCL cytokines (CXCL1-3, and 5-8) mediate their effect through their binding to the G-protein-coupled receptors CXCR1 and CXCR2, which leads to the activation of the extracellular signal-regulated kinase (ERK) and phosphoinositide 3-kinase (PI3K) pathways. The ELR⁺CXCL leading member, CXCL8 (interleukin 8 or IL-8) promotes angiogenesis, inflammation, tumorigenesis, and metastasis. Moreover, Ras-dependent secretion of CXCL8 enhances tumor progression by promoting neovascularization, and its binding to CXCR2 is involved in several cancer cell survival, such as prostate, ovarian, brain, skin, and kidney. On the other hand, CXCL1 is involved in esophageal and melanoma cancer cell proliferation and CXCL7 is involved in the development of the lymphatic network through the regulation of VEGF-C and VEGF-D in breast cancer. Few CXCR1 and CXCR2 inhibitors are currently in clinical trials, mainly for the treatment of pulmonary inflammatory disorders. Examples of CXCR antagonists already marketed or in clinical trials are for instance Reparixin, DF2156A, SCH-527123, SB-225002, SB-656933, and Danirixin (GSK-1355756). It has been reported that soluble analogs of SB225002 (a 2-hydroxyphenylurea derivative developed by GSK company) inhibit tumor growth, angiogenesis and inflammation in vitro and in vivo in clear cell renal cell carcinoma model (786-O xenograft mice) by antagonizing the effect of pro-inflammatory cytokines CXCL 1, 7 and 8, underlining thereby the CXCL1,7,8/CXCR1/CXCR2 axis as a pertinent target for the treatment of the chronic angiogenesis and inflammation observed in cancers.

Therefore, there is a need for developing new antagonists targeting CXCR1/CXCR2 receptors and able to tackle concomitantly inflammation and angiogenesis in order to treat cancer and/or disorders characterized by undesirable excessive angiogenesis, such as macular degeneration.

### SUMMARY OF THE INVENTION

In this context, the inventors have surprisingly identified and demonstrated that CXCR1 and CXCR2 receptors antagonists of formula (I) are useful for treating a cancer by acting on three major hallmarks of cancers. Indeed, the compounds of formula (I), and more particularly compound #1, exert a dual activity on both angiogenesis and inflammation in addition to reduce tumour growth. The inventors have further surprisingly identified that CXCR1 and CXCR2 receptors antagonists of formula (I) are useful for treating macular degeneration, particularly compounds #3 and #1.

The present invention thus relates to a compound, a pharmaceutically acceptable salt or a tautomer thereof, of formula (II):

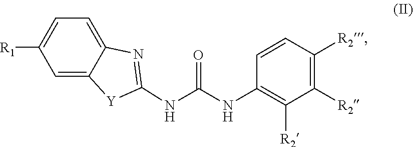

in which:
Y is NH or S;
R₁ is a radical selected in the group consisting of a hydrogen atom, nitro group, a (C₁-C₆)alkyl group, and a (C₁-C₆)alkyloxy group;
R_{2′}, R_{2″}, and R_{2′″} represent independently a hydrogen atom, a halogen atom, a (C₁-C₆)alkyl group, or a (C₁-C₆)alkyloxy group;
   for use for treating macular degeneration.

In a particular embodiment, macular degeneration is a wet macular degeneration or a dry macular degeneration, preferably a wet macular degeneration.

In a further particular embodiment, Y is S and R₁ is a (C₁-C₆)alkyloxy group.

A preferred compound of formula (II) for use for treating macular degeneration, particularly a wet or dry macular degeneration, is 1-(6-ethoxybenzo[d]thiazol-2-yl)-3-(o-tolyl)urea.

The present invention further relates to a compound, a pharmaceutically acceptable salt or a tautomer thereof, of formula (I):

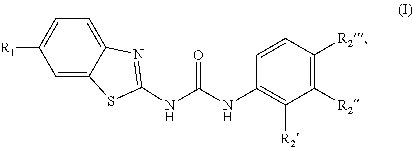

in which:
R₁ is a radical selected in the group consisting of a nitro group, a (C₁-C₆)alkyl group, and a (C₁-C₆)alkyloxy group;
R_{2′}, R_{2″}, and R_{2′″} represent independently a hydrogen, a halogen, or a (C₁-C₆)alkyl group, wherein two substituents chosen among R_{2′}, R_{2″}, and R_{2′″} are a hydrogen and the other is a halogen or a (C₁-C₆)alkyl group; and
   with the proviso that the compound of formula (I) is not a compound selected in the group consisting of:
1-(4-chlorophenyl)-3-(6-methoxybenzo[d]thiazol-2-yl)urea;
1-(3-fluorophenyl)-3-(6-methoxybenzo[d]thiazol-2-yl)urea;
1-(6-nitrobenzo[d]thiazol-2-yl)-3-o-tolylurea; and
1-(6-nitrobenzo[d]thiazol-2-yl)-3-m-tolylurea;
   for use for treating a cancer.

In a particular embodiment, R₁ is a radical selected in the group consisting of a nitro group, a methyl group, and an ethoxy group.

In a further particular embodiment, R_{2′}, R_{2″}, and R_{2′″} represent independently a hydrogen, a chlorine atom, a bromine atom, or a methyl group, wherein two substituents chosen among R_{2′}, R_{2″}, and R_{2′″} are a hydrogen and the other is a chorine atom, a bromine atom or a methyl group.

In a preferred embodiment, R₁ is a nitro group, and R_{2′}, R_{2″}, and R_{2′″} represent independently a hydrogen, a chlorine atom or a bromine atom, wherein two substituents chosen among R_{2′}, R_{2″}, and R_{2′″} are a hydrogen and the other is a chlorine atom or a bromine atom.

A preferred compound of formula (I) for use for treating a cancer is a compound selected in the group consisting of:
1-(3-chlorophenyl)-3-(6-nitrobenzo[d]thiazol-2-yl)urea;
1-(2-chlorophenyl)-3-(6-nitrobenzo[d]thiazol-2-yl)urea;
1-(6-ethoxybenzo[d]thiazol-2-yl)-3-(o-tolyl)urea;
1-(2-chlorophenyl)-3-(6-methylbenzo[d]thiazol-2-yl)urea;
1-(4-bromophenyl)-3-(6-nitrobenzo[d]thiazol-2-yl)urea; and
1-(2-bromophenyl)-3-(6-nitrobenzo[d]thiazol-2-yl)urea.

In a further preferred embodiment, the cancer is a medulloblastoma, a head and neck cancer, a kidney cancer, or a triple-negative breast cancer.

In a particular embodiment, the present invention relates to a compound of formula (I) as defined herein for use for treating a head and neck cancer in a subject resistant to cisplatin, oxaliplatin, or carboplatin, preferably cisplatin.

In a further particular embodiment, the present invention relates to a compound of formula (I) as defined herein for use for treating a kidney cancer in a subject resistant to Sunitinib, Axitinib, or Cabozantinib, preferably Sunitinib.

A further object of the invention is a pharmaceutical composition comprising a compound of formula (I) or a pharmaceutically acceptable salt thereof as defined herein, for use in the treatment of a cancer.

In a particular embodiment, said composition is administered at a dose from 1 to 1000 mg/kg BW, preferably from 10 to 250 mg/kg BW, more preferably from 50 to 100 mg/kg BW.

In a further particular embodiment, said composition is administered by oral or parenteral route, preferably by intraperitoneal route.

A further object of the invention is a compound, a pharmaceutically acceptable salt or a tautomer thereof, of formula (II):

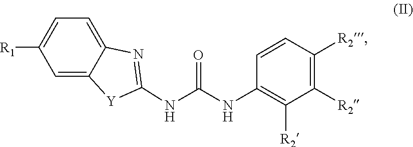

in which:
Y is NH or S;
R₁ is a radical selected in the group consisting of a hydrogen atom, nitro group, a (C₁-C₆)alkyl group, and a (C₁-C₆)alkyloxy group;
R_{2′}, R_{2″}, and R_{2′″} represent independently a hydrogen atom, a halogen atom, a (C₁-C₆)alkyl group, or a (C₁-C₆)alkyloxy group;
   for use for treating a cancer selected in the group consisting of a medulloblastoma, a head and neck cancer, and a kidney cancer.

A preferred compound of formula (II) for use for treating a cancer selected in the group consisting of a medulloblastoma, a head and neck cancer, and a kidney cancer is a compound selected in the group consisting of:
1-(3,5-dichlorophenyl)-3-(6-nitrobenzo[d]thiazol-2-yl)urea; and
1-(3-chlorophenyl)-3-(6-nitro-1H-benzo[d]imidazole-2-yl)urea.

A further object of the invention is a compound, a salt or a tautomer thereof, selected in the group consisting of:
1-(2-chlorophenyl)-3-(6-nitrobenzo[d]thiazol-2-yl)urea;
1-(6-ethoxybenzo[d]thiazol-2-yl)-3-(o-tolyl)urea;
1-(2-chlorophenyl)-3-(6-methylbenzo[d]thiazol-2-yl)urea;
1-(3,5-dichlorophenyl)-3-(6-nitrobenzo[d]thiazol-2-yl)urea;
1-(4-bromophenyl)-3-(6-nitrobenzo[d]thiazol-2-yl)urea;
1-(2-bromophenyl)-3-(6-nitrobenzo[d]thiazol-2-yl)urea; and
1-(3-chlorophenyl)-3-(6-nitro-1H-benzo[d]imidazole-2-yl)urea.

Another object is a pharmaceutical comprising such compound and a pharmaceutically acceptable carrier. A further object is such compound for use as a medicine.

### LEGEND OF FIGURES

FIG. 1: Observation by FACS analyses of early (Annexin AV) and late (Propidium Iodide, PI) apoptosis markers expressed by healthy and malignant cells after treatment with compounds #1 and #2 at 5 μM. CT: negative control.

FIG. 2: Compound #1 exerts cytotoxic and cytostatic effects against sensitive and sunitinib-resistant 786-O cells;

A, B: Naive (786-O, A), and sunitinib-resistant (786-R, B) 786-O cells, were treated with compound #1 or sunitinib (1 to 10 μM) for 48 hr. Cell viability was measured by XTT assays;

C: 786 and 786-R cells were treated with 2.5 μM compound #1 for 48 h. Cells were stained with the PI/annexin-V-fluo staining kit according to the manufacturer's indications. Histograms show both annexin-V⁺/PI⁻ cells (blue bars) and annexin-V⁺/PI⁺ cells (red bars);

D, E: 786-O (D), 786-R (E), were treated with 2.5 μM compound #1 or 2.5 μM sunitinib for 24 h to 96 h. The cells metabolism was measured by XTT assay;

F: Cells were treated with 2.5 and 5 μM compound #1, with 2.5 μM sunitinib for 48 hr. Cells were lysed in caspase buffer and caspase-3 activity was evaluated using 0.2 mM Ac-DEVD-AMC as substrates. Results expressed as arbitrary units (A.U.) and are means±Standard deviation of 3 independent experiments;

G: Clonogenicity assays: RCC cells (768 and 786-R) were treated with compound #1 or sunitinib (1 μM) and colored with Giemsa blue after 10 days (representative results of an experiment repeated trice);

H: 786-O and 786-R were treated with 2.5 μM compound #1 for 1 to 48 h. p-ERK and p-AKT levels were determined by immunoblotting. ERK and AKT served as loading controls. *P<0.05; **P<0.01; ***P<0.001.

FIG. 3: Compound #1 exerts cytotoxic effects against sensitive and cisplatin-resistant Cal27 cells;

A, B: Naive (Cal27, A), and cisplatine-resistant (Cal27R, B) Cal27 cells, were treated with compound #1 or cisplatine (1 to 10 μM) for 48 hr. Cell viability was measured by XTT assays;

C, D: Cal27 (C), cal27R (D), were treated with 2.5 μM compound #1, 2.5 μM cisplatin for 24 h to 96 h. The metabolism of cells was measured by XTT assay.

FIG. 4: Compound #1 exerts cytotoxic effects against primary RCC cells;

A: Primary RCC cells (TF and CC) and normal renal cells (15S) were treated with compound #1 (1 to 5 μM) for 48 h. Cell viability was measured by XTT assays;

B: Primary RCC cells (TF and CC) were treated with compound #1 (1 or 2.5 μM) and colored with Giemsa blue after 10 days (representative results of an experiment repeated trice);

C: Primary RCC cells (TF and CC) and normal renal cells (15S) were treated with compound #1 (1 to 5 μM) for 48 h. Cells were stained with the PI/annexin-V-fluos staining kit according to the manufacturer's indications. Histograms show both annexin-V+/PI− cells (open bars) and annexin-V+/PI+ cells (filled bars). *P<0.05; **P<0.01; ***P<0.001.

FIG. 5: Compound #1 inhibits the ERL+CXCL/CXCR2 axis in endothelial cells;

A: HuVEC were stimulated with 25 ng/ml CXCL8 during 1 h. Membrane-associated CXCR2 protein levels were quantified by flow cytometry;

B: CXCL7 (50 ng/ml) or VEGFA (50 ng/ml)-dependent HuVEC migration was analyzed using Boyden chamber assays in presence/absence of compound #1 or danirixin;

C: HuVEC were grown in the presence of different concentrations of compound #1 for 48h. Cell viability was measured by XTT assays;

D: HuVEC were incubated with 100 ng/ml CXCL7 or CXCL5, in presence of 0.25 or 0.5 μM compound #1 for 48 h. Cell viability was measured by XTT assays;

E: HuVEC were pre-treated with 5 μM compound #1 for 1 h then stimulated with 50 ng/ml CXCL5 for 10 min. p-ERK levels were analyzed by immunoblotting. ERK and HSP60 served as loading controls *P<0.05; **P<0.01; ***P<0.001.

FIG. 6: In vivo mouse xenograft experiments;

A: The tumor volume was measured twice weekly as described in materials and methods. The results are presented as the means±SD;

B: At the end of experiments, tumors were weighted;

C: Weight of the animals at the end of the experiment (day 70);

D: Human Ki-67 expression of untreated and treated mice. The number of proliferative cells was determined by calculating the ratio of colocalized 4,6 diamidino-2-phenylindole (DAPI)/Ki-67-positive cells with respect to total cell number;

E and F: the levels of pERK, ERK, pAKT and AKT in tumor lysates were determined by immunoblotting. The graphs represent the ratio of pAKT (F) or pERK (E) to non-phosphorylated ERK or AKT;

G: The level of murine CD31 mRNA in tumors were measured by qPCR; H, I, J, K: The levels of human VEGFA, CXCL5, CXCL7 and CXCL8 mRNA in tumors were evaluated by qPCR.

FIG. 7: Inhibition of proliferation of medulloblastoma cells by compound #1. Cells were treated or not with 1 μM compound #1 for the indicated days. Cells were counted at the indicated days. * p<0.05; ** p<0.01; *** p<0.001.

FIG. 8: Inhibition of proliferation of medulloblastoma cells by compound #1. Clonogenicity tests; *** p>0.001.

FIG. 9: Evaluation of compounds #1 and #3 on macular degeneration. Clinical angiography at Day 14: Evaluation of the intensity of the lesion by a score from 0 to 3 (0: no leak; 1: light intensity; 2: moderate intensity; 3: immense marking) on mice treated with compounds #1 and #3. The intensities of the lesions were reported, each point corresponding to a lesion. Three lesions were performed on the eyes of each mouse; * p>0.05.

### DETAILED DESCRIPTION OF THE INVENTION

The inventors have identified that compounds of formulae (I) and (II) have a therapeutic interest for treating cancer as antagonists of CXCR1/CXCR2 receptors. Such compounds are also interesting for treating disorders characterized by undesirable excessive angiogenesis. As demonstrated by the examples, such compounds have a triple anti-cancer activity by exerting an effect against angiogenesis, inflammation, and the growth of tumors. Such compounds are thus particularly suitable for treating a cancer in which CXCR1 and CXCR2 are overexpressed, for instance a medulloblastoma, a head and neck cancer, a kidney cancer, and a triple-negative breast. The inventors have further surprisingly identified that the compounds of the invention, more particularly compound #1, was found to be significantly active against cancer cells resistant to conventional drugs sunitinib and cisplatine, which are the current golden standard of care for kidney cancer and head and neck cancer, respectively. The inventors have also surprisingly identified that the compounds of the invention, more particularly compounds #3 and #1, have a therapeutic interest for treating macular degeneration.

According to the present invention, the terms below have the following meanings: The compounds of formulae (I) and (II) include the pharmaceutically acceptable salts thereof as well as their tautomers, enantiomers, diastereoisomers, racemates of mixtures thereof, hydrates and solvates. Particularly, the compounds of formulae (I) and (II) include the tautomers thereof. A tautomer of a compound of formula (I) may have the following formula:

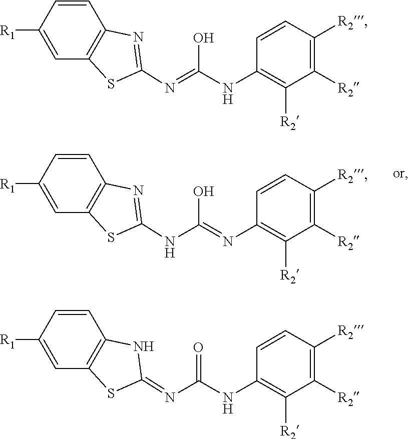

with R₁, R_{2′}, R_{2″}, and R_{2′″} are such as defined herein. A tautomer of a compound of formula (II) may have the following formula:

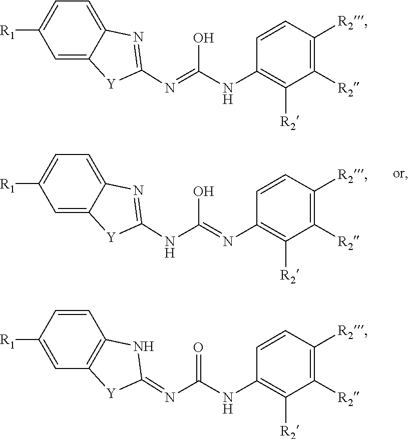

with Y, R₁, R_{2′}, R_{2″}, and R_{2′″} are such as defined herein. The terms mentioned herein with prefixes such as for example C₁-C₃ or C₁-C₆ can also be used with lower numbers of carbon atoms such as C₁-C₂, or C₁-C₅. If, for example, the term C₁-C₃ is used, it means that the corresponding hydrocarbon chain may comprise from 1 to 3 carbon atoms, especially 1, 2 or 3 carbon atoms. If, for example, the term C₁-C₆ is used, it means that the corresponding hydrocarbon chain may comprise from 1 to 6 carbon atoms, especially 1, 2, 3, 4, 5 or 6 carbon atoms.

The term “alkyl” refers to a saturated, linear or branched aliphatic group. The term “(C₁-C₃)alkyl” more specifically means methyl, ethyl, propyl, or isopropyl. The term “(C₁-C₆)alkyl” more specifically means methyl, ethyl, propyl, isopropyl, butyl, isobutyl, tert-butyl, pentyl or hexyl. In a preferred embodiment, the “alkyl” is a methyl, an ethyl, a propyl, an isopropyl, or a tert-butyl, more preferably a methyl.

The term “alkyloxy” or “alkoxy” corresponds to the alkyl group as above defined bonded to the molecule by an —O— (ether) bond. (C₁-C₃)alkyloxy includes methoxy, ethoxy, propyloxy, and isopropyloxy. (C₁-C₆)alkyloxy includes methoxy, ethoxy, propyloxy, isopropyloxy, butyloxy, isobutyloxy, tert-butyloxy, pentyloxy and hexyloxy. In a preferred embodiment, the “alkoxy” or “alkyloxy” is an ethoxy.

The term “halogen” corresponds to a fluorine, a chlorine, a bromine, or an iodine atom, preferably a chlorine or a bromine atom, more preferably a chlorine.

As used herein, the term “pharmaceutically acceptable salt” includes inorganic as well as organic acids salts. Representative examples of suitable inorganic acids include hydrochloric, hydrobromic, hydroiodic, phosphoric, and the like. Representative examples of suitable organic acids include formic, acetic, trichloroacetic, trifluoroacetic, propionic, benzoic, cinnamic, citric, fumaric, maleic, methanesulfonic and the like. Further examples of pharmaceutically acceptable inorganic or organic acid addition salts include the pharmaceutically acceptable salts listed in J. Pharm. Sci. 1977, 66, 2, and in Handbook of Pharmaceutical Salts: Properties, Selection, and Use edited by P. Heinrich Stahl and Camille G. Wermuth 2002. In a preferred embodiment, the salt is a hydrochloride salt.

Compounds

The present invention thus relates to a compound, a pharmaceutically acceptable salt or a tautomer thereof, of formula (I):

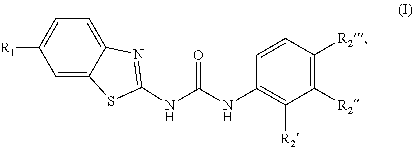

in which:

R₁ is a radical selected in the group consisting of a nitro group, a (C₁-C₆)alkyl group, and a (C₁-C₆)alkyloxy group;

R_{2′}, R_{2″}, and R_{2′″} represent independently a hydrogen, a halogen, or a (C₁-C₆)alkyl group, wherein two substituents chosen among R_{2′}, R_{2″}, and R_{2′″} are a hydrogen and the other is a halogen or a (C₁-C₆)alkyl group; and with the proviso that the compound of formula (I) is not a compound selected in the group consisting of:
1-(4-chlorophenyl)-3-(6-methoxybenzo[d]thiazol-2-yl)urea;
1-(3-fluorophenyl)-3-(6-methoxybenzo[d]thiazol-2-yl)urea;
1-(6-nitrobenzo[d]thiazol-2-yl)-3-o-tolylurea; and
1-(6-nitrobenzo[d]thiazol-2-yl)-3-m-tolylurea;
for use for treating a cancer.

In one particular embodiment, a compound of formula (I) is such that R₁ is a nitro group.

In one particular embodiment, a compound of formula (I) is such that R₁ is a (C₁-C₆)alkyl group. In one particular embodiment, a compound of formula (I) is such that R₁ is a (C₁-C₆)alkyloxy group.

In a further particular embodiment, a compound of formula (I) is such that R₁ is a radical selected in the group consisting of a nitro group, a methyl group, and an ethoxy group.

As above defined, a compound of formula (I) for use is such that R_{2′}, R_{2″}, and R_{2′″} represent independently a hydrogen, a halogen, or a (C₁-C₆)alkyl group, wherein two substituents chosen among R_{2′}, R_{2″}, and R_{2′″} are a hydrogen and the other is a halogen or a (C₁-C₆)alkyl group. In a particular embodiment, R_{2′}, R_{2″}, and R_{2′″} represent independently a hydrogen, a chlorine atom a bromine atom, or a methyl group, wherein two substituents chosen among R_{2′}, R_{2″}, and R_{2′″} are a hydrogen and the other is a chorine atom, a bromine atom or a methyl group.

The expression “two substituents chosen among R_{2′}, R_{2″}, and R_{2′″} are a hydrogen and the other is a halogen or a (C₁-C₆)alkyl group particularly means that:
R_{2′} and R_{2″} are a hydrogen atom and R_{2′″} is a halogen, preferably a chlorine or a bromine, or a (C₁-C₆)alkyl group, preferably a methyl group;
R_{2′} and R_{2″} are a hydrogen atom and R_{2′″} is a halogen, preferably a chlorine or a bromine, or a (C₁-C₆)alkyl group, preferably a methyl group; and
R_{2′} and R_{2″} are a hydrogen atom and R_{2′″} is a halogen, preferably a chlorine or a bromine, or a (C₁-C₆)alkyl group, preferably a methyl group.

In a preferred embodiment, a compound of formula (I) for use is such that:
R₁ is a nitro group; and
R_{2′}, R_{2″}, and R_{2′″} represent independently a hydrogen, a chlorine atom or a bromine atom, wherein two substituents chosen among R_{2′}, R_{2″}, and R_{2′″} are a hydrogen and the other is a chlorine atom or a bromine atom.

In a further preferred embodiment, a compound of formula (I) for use is selected in the group consisting of:
1-(3-chlorophenyl)-3-(6-nitrobenzo[d]thiazol-2-yl)urea;
1-(2-chlorophenyl)-3-(6-nitrobenzo[d]thiazol-2-yl)urea;
1-(6-ethoxybenzo[d]thiazol-2-yl)-3-(o-tolyl)urea;
1-(2-chlorophenyl)-3-(6-methylbenzo[d]thiazol-2-yl)urea;
1-(4-bromophenyl)-3-(6-nitrobenzo[d]thiazol-2-yl)urea; and
1-(2-bromophenyl)-3-(6-nitrobenzo[d]thiazol-2-yl)urea.

The present invention further relates to a compound, a pharmaceutically acceptable salt or a tautomer thereof, of formula (II):

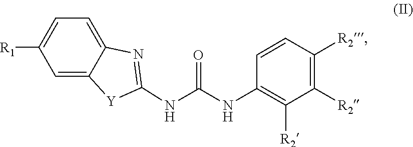

in which:
Y is NH or S;
R₁ is a radical selected in the group consisting of a hydrogen atom, a nitro group, a (C₁-C₆)alkyl group, and a (C₁-C₆)alkyloxy group;
R_{2′}, R_{2″}, and R_{2′″} represent independently a hydrogen atom, a halogen atom, a (C₁-C₆)alkyl group, or a (C₁-C₆)alkyloxy group;
   for use for treating a cancer selected in the group consisting of a medulloblastoma, a head and neck cancer and a kidney cancer.

In a particular embodiment, a compound of formula (II) for use is such that:
Y is NH or S;
R₁ is a radical selected in the group consisting of a hydrogen atom, a nitro group, a methyl group, and an ethoxy group;
R_{2′}, R_{2″}, and R_{2′″} represent independently a hydrogen atom, a chlorine atom, a methyl group, or a methoxy group.

In a further particular embodiment, a compound of formula (II) for use is such that:
Y is NH or S;
R₁ is a hydrogen atom;
R_{2′}, R_{2″}, and R_{2′″} represent independently a hydrogen atom, a chlorine atom, or a methoxy group.

In a further particular embodiment, a compound of formula (II) for use is such that:
Y is NH or S;
R₁ is a nitro group;
R_{2′}, R_{2″}, and R_{2′″} represent independently a hydrogen atom or a chlorine atom.

In a preferred embodiment, a compound of formula (II) for use is such that R_{2′}, R_{2″}, and R_{2′″} represent independently a hydrogen atom, a halogen, preferably a chlorine atom, a (C₁-C₆)alkyl group, preferably a methyl group, or a (C₁-C₆)alkyloxy group, preferably a methoxy group, wherein two substituents chosen among R_{2′}, R_{2″}, and R_{2′″} are a hydrogen atom and the other is a halogen, preferably a chlorine atom, a (C₁-C₆)alkyl group, preferably a methyl group, or a (C₁-C₆)alkyloxy group, preferably a methoxy group.

In a further preferred embodiment, a compound of formula (II) for use is such that R_{2′}, R_{2″}, and R_{2′″} represent independently a hydrogen atom or a halogen, wherein one or two substituents chosen among R_{2′}, R_{2″}, and R_{2′″} are a hydrogen atom and the other or the two others are a halogen, preferably a chlorine atom.

In a more preferred embodiment, a compound of formula (II) for use is selected in the group consisting of:
1-(3-chlorophenyl)-3-(6-nitrobenzo[d]thiazol-2-yl)urea;
1-(2-chlorophenyl)-3-(6-nitrobenzo[d]thiazol-2-yl)urea;
1-(6-ethoxybenzo[d]thiazol-2-yl)-3-(o-tolyl)urea;
1-(2-chlorophenyl)-3-(6-methylbenzo[d]thiazol-2-yl)urea;
1-(1H-benzo[d]imidazol-2-yl)-3-phenylurea;
1-(1H-benzo[d]imidazol-2-yl)-3-(4-chlorophenyl)urea;
1-(benzo[d]thiazol-2-yl)-3-(2-chlorophenyl)urea;
1-(benzo[d]thiazol-2-yl)-3-(3-chlorophenyl)urea;
1-(benzo[d]thiazol-2-yl)-3-(4-chlorophenyl)urea; and
1-(benzo[d]thiazol-2-yl)-3-(4-methoxyphenyl)urea.

In an even more preferred embodiment, a compound of formula (II) for use is selected in the group consisting of:
1-(3,5-dichlorophenyl)-3-(6-nitrobenzo[d]thiazol-2-yl)urea; and
1-(3-chlorophenyl)-3-(6-nitro-1H-benzo[d]imidazole-2-yl)urea.

The present invention further relates to a compound, a salt or a tautomer thereof, selected in the group consisting of:
1-(2-chlorophenyl)-3-(6-nitrobenzo[d]thiazol-2-yl)urea;
1-(6-ethoxybenzo[d]thiazol-2-yl)-3-(o-tolyl)urea;
1-(2-chlorophenyl)-3-(6-methylbenzo[d]thiazol-2-yl)urea;
1-(3,5-dichlorophenyl)-3-(6-nitrobenzo[d]thiazol-2-yl)urea;
1-(4-bromophenyl)-3-(6-nitrobenzo[d]thiazol-2-yl)urea;
1-(2-bromophenyl)-3-(6-nitrobenzo[d]thiazol-2-yl)urea; and
1-(3-chlorophenyl)-3-(6-nitro-1H-benzo[d]imidazole-2-yl)urea.

The present invention also relates to N—N′-diarylureas and thioureas for use for treating a cancer.

More particularly, a further object of the invention is a compound or a pharmaceutically acceptable salt thereof of formula (III):

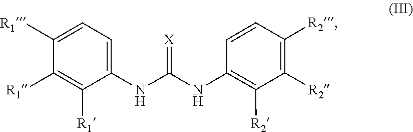

in which:
X is O or S;
R_{2′}, R_{2″}, and R_{2′″} represent independently a hydrogen atom, a (C₁-C₆)alkyl group, a halogen, or a hydroxy group; and
R_{2′}, R_{2″}, and R_{2′″} represent independently a hydrogen atom or a halogen;
   for use for treating cancer, preferably a medulloblastoma, a head and neck cancer or a kidney cancer.

In a particular embodiment, a compound of formula (III) for use is such that:
X is O or S, preferably O;
R_{2′}, R_{2″}, and R_{2′″} represent independently a hydrogen atom, a methyl group, a chlorine atom, or a hydroxy group; and
R_{2′}, R_{2″}, and R_{2′″} represent independently a hydrogen or a chlorine atom, wherein two substituents chosen among R_{2′}, R_{2″}, and R_{2′″} are a hydrogen atom and the other is a chlorine atom;
   for use for treating a cancer, preferably medulloblastoma, a head and neck cancer or a kidney cancer.

In a preferred embodiment, a compound of formula (III) for use is selected in the group consisting of:
1-(2-chlorophenyl)-3-(p-tolyl)urea;
1-(3-chlorophenyl)-3-(p-tolyl)urea;
1-(4-chlorophenyl)-3-(p-tolyl)urea;
1-(2-chlorophenyl)-3-(2,4-dichlorophenyl)urea;
1-(3-chlorophenyl)-3-(2,4-dichlorophenyl)urea;
1-(4-chlorophenyl)-3-(2,4-dichlorophenyl)urea;
1-phenyl-3-(p-tolyl)thiourea;
1-(2,4-dichlorophenyl)-3-phenylthiourea;
1,3-bis(3-chlorophenyl)urea;
1,3-bis(4-chlorophenyl)urea;
1-(3-chlorophenyl)-3-(2-hydroxyphenyl)urea; and
1-(4-chlorophenyl)-3-(2-hydroxyphenyl)urea.

Thanks to their capacity to regulate angiogenesis, the compounds of the invention may be used for the treatment of disorders characterized by undesirable excessive angiogenesis, such as macular degeneration, and more preferably, age-related macular degeneration.

A further object of the invention is thus a compound of formula (I) or (II) as above defined for use for treating a disorder characterized by undesirable excessive angiogenesis such as macular degeneration, in particular, age-related macular degeneration.

A particular object of the invention is thus a compound, a pharmaceutically acceptable salt or a tautomer thereof of formula (II):

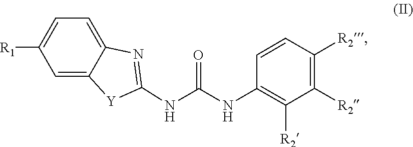

in which:
Y is NH or S;
R₁ is a radical selected in the group consisting of a hydrogen atom, nitro group, a (C₁-C₆)alkyl group, and a (C₁-C₆)alkyloxy group;
R_{2′}, R_{2″}, and R_{2′″} represent independently a hydrogen atom, a halogen atom, a (C₁-C₆)alkyl group, or a (C₁-C₆)alkyloxy group;
   for use for treating macular degeneration.

In a particular embodiment, macular degeneration is a wet macular degeneration or a dry macular degeneration. Preferably, macular degeneration is a wet macular degeneration.

In a further particular embodiment, macular degeneration is an age-related macular degeneration. Preferably macular degeneration is an age-related wet macular degeneration

In a preferred embodiment, a compound of formula (II) for use for treating macular degeneration is such that Y is S.

In a further preferred embodiment, a compound of formula (II) for use for treating macular degeneration is such that R₁ is a (C₁-C₆)alkyloxy group, preferably an ethoxy.

In a more preferred embodiment, a compound of formula (II) for use for treating macular degeneration is such that Y is S, and R₁ is a (C₁-C₆)alkyloxy group, preferably an ethoxy.

In a further preferred embodiment, a compound of formula (II) for use for treating a disorder characterized by undesirable excessive angiogenesis, in particular macular degeneration and more preferably age-related macular degeneration, is selected in the group consisting of:
1-(3-chlorophenyl)-3-(6-nitrobenzo[d]thiazol-2-yl)urea;
1-(2-chlorophenyl)-3-(6-nitrobenzo[d]thiazol-2-yl)urea;
1-(6-ethoxybenzo[d]thiazol-2-yl)-3-(o-tolyl)urea;
1-(2-chlorophenyl)-3-(6-methylbenzo[d]thiazol-2-yl)urea;
1-(1H-benzo[d]imidazol-2-yl)-3-phenylurea;
1-(1H-benzo[d]imidazol-2-yl)-3-(4-chlorophenyl)urea;
1-(benzo[d]thiazol-2-yl)-3-(2-chlorophenyl)urea;
1-(benzo[d]thiazol-2-yl)-3-(3-chlorophenyl)urea;
1-(benzo[d]thiazol-2-yl)-3-(4-chlorophenyl)urea;
1-(benzo[d]thiazol-2-yl)-3-(4-methoxyphenyl)urea;
1-(3,5-dichlorophenyl)-3-(6-nitrobenzo[d]thiazol-2-yl)urea;
1-(4-bromophenyl)-3-(6-nitrobenzo[d]thiazol-2-yl)urea;
1-(2-bromophenyl)-3-(6-nitrobenzo[d]thiazol-2-yl)urea; and
1-(3-chlorophenyl)-3-(6-nitro-1H-benzo[d]imidazole-2-yl)urea.

In a more preferred embodiment, such compound is 1-(6-ethoxybenzo[d]thiazol-2-yl)-3-(o-tolyl)urea.

A further particular object of the invention is a compound or a pharmaceutically acceptable salt thereof of formula (I):

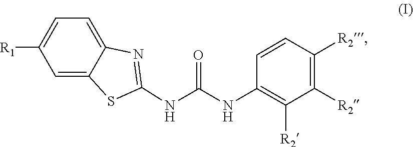

in which:
R₁ is a radical selected in the group consisting of a nitro group, a (C₁-C₆)alkyl group, and a (C₁-C₆)alkyloxy group;
R_{2′}, R_{2″}, and R_{2′″} represent independently a hydrogen, a halogen, or a (C₁-C₆)alkyl group, wherein two substituents chosen among R_{2′}, R_{2″}, and R_{2′″} are a hydrogen and the other is a halogen or a (C₁-C₆)alkyl group; and
   with the proviso that the compound of formula (I) is not a compound selected in the group consisting of:
1-(6-nitrobenzo[d]thiazol-2-yl)-3-o-tolylurea; and
1-(6-nitrobenzo[d]thiazol-2-yl)-3-m-tolylurea;
   for use for treating a disorder characterized by undesirable excessive angiogenesis, in particular macular degeneration, and more preferably age-related macular degeneration.

In a particular embodiment, a compound or a pharmaceutically acceptable salt thereof of formula (I) for use for treating macular generation is such that R₁ is a (C₁-C₆)alkyloxy group and R₂, R_{2″}, and R_{2′″} represent independently a hydrogen, a halogen, or a (C₁-C₆)alkyl group, wherein two substituents chosen among R_{2′}, R_{2″}, and R₂″ are a hydrogen and the other is a halogen or a (C₁-C₆)alkyl group.

A preferred object of the invention is a compound or a salt thereof selected in the group consisting of:
1-(3-chlorophenyl)-3-(6-nitrobenzo[d]thiazol-2-yl)urea;
1-(2-chlorophenyl)-3-(6-nitrobenzo[d]thiazol-2-yl)urea;
1-(6-ethoxybenzo[d]thiazol-2-yl)-3-(o-tolyl)urea; and
1-(2-chlorophenyl)-3-(6-methylbenzo[d]thiazol-2-yl)urea;
   preferably 1-(6-ethoxybenzo[d]thiazol-2-yl)-3-(o-tolyl)urea;

for use for treating a disorder characterized by undesirable excessive angiogenesis, in particular macular degeneration and more preferably age-related macular degeneration.

### Application

According to the present invention, the terms below have the following meanings:

As used herein, the terms “treatment”, “treat” or “treating” refer to any act intended to ameliorate the health status of patients such as therapy, prevention, prophylaxis and retardation of a disease.

In certain embodiments, such terms refer to the amelioration or eradication of the disease, or symptoms associated with it. In other embodiments, this term refers to minimizing the spread or worsening of the disease, resulting from the administration of one or more therapeutic agents to a subject with such a disease.

As used herein, the terms “subject”, “individual” or “patient” are interchangeable and refer to an animal, preferably to a mammal, even more preferably to a human.

The terms “quantity,” “amount,” and “dose” are used interchangeably herein and may refer to an absolute quantification of a molecule.

As used herein, the terms “active principle”, “active ingredient” and “active pharmaceutical ingredient” are equivalent and refer to a component of a pharmaceutical composition having a therapeutic effect. Particularly, such terms designate a compound of formula (I) or (II).

As used herein, the term “therapeutic effect” refers to an effect induced by an active ingredient, or a pharmaceutical composition according to the invention, capable to prevent or to delay the appearance or development of a disease or disorder, or to cure or to attenuate the effects of a disease or disorder, particularly a cancer or a disorder characterized by undesirable excessive angiogenesis, such as macular degeneration.

As used herein, the term “effective amount” refers to a quantity of an active ingredient or of a pharmaceutical composition that prevents, removes or reduces the deleterious effects of the disease, particularly a cancer or a disorder characterized by undesirable excessive angiogenesis. It is obvious that the quantity to be administered can be adapted by the man skilled in the art according to the subject to be treated, to the nature of the disease, etc. In particular, doses and regimen of administration may be adapted to the nature, the stage and the severity of the disease to be treated, as well as the weight, the age and the global health of the subject to be treated, as well as the judgment of the doctor.

As used herein, the term “excipient or pharmaceutically acceptable carrier” refers to any ingredient except active ingredients that is present in a pharmaceutical composition. Its addition may be aimed to confer a particular consistency or other physical or gustative properties to the final product. An excipient or pharmaceutically acceptable carrier must be devoid of any interaction, in particular chemical, with the active ingredients.

As used herein, the term “cancer” refers to the presence of cells possessing characteristics typical of cancer-causing cells, such as uncontrolled proliferation, immortality, metastatic potential, rapid growth and proliferation rate, and certain characteristic morphological features. The cancer may be solid tumors or hematopoietic tumors. More specifically, the cancer is a cancer overexpressing the CXCR1 and CXCR2 receptors such as leukemia, kidney cancer, medulloblastoma, head and neck cancer, and triple-negative breast cancer. The expression “triple-negative breast cancer” refers to a breast cancer that does not express the genes for estrogen receptor (ER), progesterone receptor (PR) and HER2/neu. In a preferred embodiment, the cancer is a kidney cancer, a medulloblastoma, a head and neck cancer, or a triple-negative breast cancer, preferably a kidney cancer or a head and neck cancer, more preferably a kidney cancer.

As used herein, the expression “disorder characterized by undesirable excessive angiogenesis” means undesirable excessive (neo)vascularization or undesirable vascular permeability. It means in particular abnormally increased angiogenesis. More specifically, a disorder characterized by undesirable excessive angiogenesis includes, without limitation, hemangioma, angiofibroma, vascular malformation, arteriosclerosis, scleroderma; ocular diseases associated with angiogenesis such as corneal graft angiogenesis, neovascular glaucoma, diabetic retinopathy, corneal diseases induced by new blood vessels, macular degeneration or age-related macular degeneration, pterygium, retinal degeneration, retrolental fibroplasia, granular conjunctivitis; chronic inflammatory diseases such as arthritis, cutaneous diseases such as psoriasis, capillarectasia, pyogenic granuloma, seborrheic dermatitis, acne, Alzheimer's disease, and obesity. In particular, the disorder characterized by undesirable excessive angiogenesis is macular degeneration including wet and dry macular degeneration, preferably age-related macular degeneration.

The present invention relates to a compound or a pharmaceutically salt thereof of formula (I) as defined herein for use for treating a cancer.

The present invention further relates to a method for treating a cancer comprising administering in a subject in need thereof an effective amount of a compound or a pharmaceutically salt thereof of formula (I) as defined herein.

The present invention also relates to a use of a compound of formula (I) as defined herein for the manufacture of a drug, a medicament, or a pharmaceutical composition for treating a cancer.

The present invention also concerns:
a compound or a pharmaceutically salt thereof of formula (I) or (II) as defined herein, for use for treating a cancer chosen among a medulloblastoma, a head and neck cancer, a kidney cancer, or a triple-negative breast cancer, preferably a head and neck cancer or a kidney cancer, more preferably a kidney cancer;
a method for treating a cancer selected in the group consisting of a medulloblastoma, a head and neck cancer, a kidney cancer, and a triple-negative breast cancer, comprising administering in a subject in need thereof an effective amount of a compound or a pharmaceutically salt thereof of formula (I) or (II) as defined herein; and
a use of a compound of formula (I) or II as defined herein for the manufacture of a drug, a medicament, or a pharmaceutical composition for treating a cancer selected in the group consisting of a medulloblastoma, a head and neck cancer, a kidney cancer, and a triple-negative breast cancer.

The compounds of the invention of formulae (I) and (II), and more particularly compound #1, are surprisingly efficient for treating cancers in subjects resistant to currents treatments.

More particularly, the present invention thus concerns:
a compound or a pharmaceutically salt thereof of formula (I) or (II) as defined herein, for use for treating a head and neck cancer in a subject resistant to cisplatin, oxaliplatin, or carboplatin, preferably cisplatin;
a method for treating a head and neck cancer, comprising administering in a subject resistant to cisplatin, oxaliplatin, or carboplatin, preferably cisplatine, an effective amount of a compound or a pharmaceutically salt thereof of formula (I) or (II) as defined herein; and
a use of a compound of formula (I) or II as defined herein for the manufacture of a drug, a medicament, or a pharmaceutical composition for treating a head and neck cancer, in a subject resistant to cisplatin, oxaliplatin, or carboplatin, preferably cisplatin.

The present invention further concerns:
a compound or a pharmaceutically salt thereof of formula (I) or (II) as defined herein, for use for treating a kidney cancer in a subject resistant to Sunitinib, Axitinib, or Cabozantinib, preferably Sunitinib;
a method for treating a kidney cancer, comprising administering in a subject resistant to Sunitinib, Axitinib, or Cabozantinib, preferably Sunitinib, an effective amount of a compound or a pharmaceutically salt thereof of formula (I) or (II) as defined herein; and
a use of a compound of formula (I) or II as defined herein for the manufacture of a drug, a medicament, or a pharmaceutical composition for treating a kidney cancer, in a subject resistant to Sunitinib, Axitinib, or Cabozantinib, preferably Sunitinib.

A further object of the invention is a pharmaceutical composition comprising a compound of formula (I) or a pharmaceutically acceptable salt thereof as defined herein, for use for treating a cancer.

A further object of the invention is a pharmaceutical composition comprising a compound of formula (II) or a pharmaceutically acceptable salt thereof as defined herein, for use for treating a cancer selected in the group consisting of a medulloblastoma, a head and neck cancer and a kidney cancer.

The present invention further relates to a compound or a pharmaceutically salt thereof of formula (I) or (II) as defined herein for use for treating a disorder characterized by undesirable excessive angiogenesis, in particular macular degeneration, and more preferably age-related macular degeneration.

The present invention further relates to a method for treating a disorder characterized by undesirable excessive angiogenesis, in particular macular degeneration, and more preferably age-related macular degeneration, comprising administering in a subject in need thereof an effective amount of a compound or a pharmaceutically salt thereof of formula (I) or (II) as defined herein. The present invention also relates to a use of a compound of formula (I) of (II) as defined herein for the manufacture of a drug, a medicament, or a pharmaceutical composition for treating a disorder characterized by undesirable excessive angiogenesis, in particular macular degeneration, and more preferably age-related macular degeneration.

In a particular embodiment, the pharmaceutical composition as defined herein comprises a compound of formula (I) or (II) in a dose from 1 to 1000 mg/kg BW, preferably from 10 to 250 mg/kg BW, more preferably from 50 to 100 mg/kg BW. An object of the invention is thus a pharmaceutical composition for use as disclosed herein, in which said composition is administered at a dose from 1 to 1000 mg/kg BW, preferably from 10 to 250 mg/kg BW, more preferably from 50 to 100 mg/kg BW. As used herein, the term “BW” means bodyweight.

In a particular aspect, the compounds and the pharmaceutical compositions for use of the invention can be administered 4, 5, 6 or 7 days a week during 1, 2, 3, 4, 5, 6 or 7 weeks.

Optionally, several treatment cycles can be performed, optionally with a break period between two treatment cycles, for instance of 1, 2, 3, 4 or 5 weeks.

The administration route can be topical, transdermal, oral, rectal, sublingual, intranasal, intrathecal, intratumoral or parenteral (including subcutaneous, intramuscular, intraperitoneal, intravenous and/or intradermal). Preferably, the administration route is oral or parenteral. More preferably, the administration route is intraperitoneal when it concerns the treatment of cancer. The pharmaceutical composition is adapted for one or several of the above-mentioned routes. The pharmaceutical composition is preferably administered by injection or by intravenous infusion of suitable sterile solutions, or in the form of liquid or solid doses via the alimentary canal. More preferably, the pharmaceutical composition is administered by an injection route.

The pharmaceutical composition can be formulated as solutions in pharmaceutically compatible solvents or as emulsions, suspensions or dispersions in suitable pharmaceutical solvents or vehicles, or as pills, tablets or capsules that contain solid vehicles in a way known in the art. Formulations of the present invention suitable for oral administration may be in the form of discrete units as capsules, sachets, tablets or lozenges, each containing a predetermined amount of the active ingredient; in the form of a powder or granules; in the form of a solution or a suspension in an aqueous liquid or non-aqueous liquid; or in the form of an oil-in-water emulsion or a water-in-oil emulsion. Formulations for rectal administration may be in the form of a suppository incorporating the active ingredient and carrier such as cocoa butter, or in the form of an enema. Formulations suitable for parenteral administration conveniently comprise a sterile oily or aqueous preparation of the active ingredient which is preferably isotonic with the blood of the recipient. Every such formulation can also contain other pharmaceutically compatible and nontoxic auxiliary agents, such as stabilizers, antioxidants, binders, dyes, emulsifiers or flavoring substances. The formulations of the present invention comprise an active ingredient in association with a pharmaceutically acceptable carrier, and optionally other therapeutic ingredients. The carrier must be “acceptable” in the sense of being compatible with the other ingredients of the formulations and not deleterious to the recipient thereof. The pharmaceutical compositions are advantageously applied by injection or intravenous infusion of suitable sterile solutions or as oral dosage by the digestive tract. Methods for the safe and effective administration of most of these chemotherapeutic agents are known to those skilled in the art. In addition, their administration is described in the standard literature.

Another object of the invention is a pharmaceutical composition comprising a compound or a salt thereof selected in the group consisting of:
1-(2-chlorophenyl)-3-(6-nitrobenzo[d]thiazol-2-yl)urea;
1-(6-ethoxybenzo[d]thiazol-2-yl)-3-(o-tolyl)urea;
1-(2-chlorophenyl)-3-(6-methylbenzo[d]thiazol-2-yl)urea;
1-(3,5-dichlorophenyl)-3-(6-nitrobenzo[d]thiazol-2-yl)urea;
1-(4-bromophenyl)-3-(6-nitrobenzo[d]thiazol-2-yl)urea;
1-(2-bromophenyl)-3-(6-nitrobenzo[d]thiazol-2-yl)urea; and
1-(3-chlorophenyl)-3-(6-nitro-1H-benzo[d]imidazole-2-yl)urea; and
   a pharmaceutically acceptable carrier.

A further object of the invention is a compound or a salt thereof selected in the group consisting of:
1-(2-chlorophenyl)-3-(6-nitrobenzo[d]thiazol-2-yl)urea;
1-(6-ethoxybenzo[d]thiazol-2-yl)-3-(o-tolyl)urea;
1-(2-chlorophenyl)-3-(6-methylbenzo[d]thiazol-2-yl)urea;
1-(3,5-dichlorophenyl)-3-(6-nitrobenzo[d]thiazol-2-yl)urea;
1-(4-bromophenyl)-3-(6-nitrobenzo[d]thiazol-2-yl)urea;
1-(2-bromophenyl)-3-(6-nitrobenzo[d]thiazol-2-yl)urea; and
1-(3-chlorophenyl)-3-(6-nitro-1H-benzo[d]imidazole-2-yl)urea; for use as a medicine.

### EXAMPLES

### Example A: Chemistry

### I. General Information

Methanol, ethyl acetate, diethyl ether and dichloromethane were purchased from Carlo Erba. Anhydrous DMF (99.8% stored under septum) was purchased from Sigma Aldrich. All chemicals were purchased from Aldrich, Fisher or Alfa Aesar. Thin-layer chromatography (TLC) was performed on precoated Merck 60 GF254 silica gel plates and revealed first by visualization under UV light (254 nm and 360 nm) ¹H and ¹³C NMR spectra were recorded on a Bruker Advance 200 MHz spectrometer or a Bruker Advance 400 MHz or a Bruker Advance 500 MHz. Mass spectra (ESI-MS) were recorded on a Bruker (Daltonics Esquire 3000+). HRMS spectra were recorded on a ThermoFisher Q Exactive (ESI-MS) at a resolution of 140 000 at m/z 200. The purity of compounds was further assayed by HPLC analysis on a JASCO PU-2089 apparatus with the following methods:
Method 1: Phenomenex Jupiter C_{18, 5} μm 250×300 mm 300A. UV-detection: 214; 254; 280; 320 nm. Eluent A: water 100%. Eluent B: CH₃CN 100%. Gradient: isocratic at 30% B for 5 minutes, then a ramp from 30% B to 90% B over 30 min, then return to initial conditions within 1 min.
Method 2: Supelco analytical column Ascentis Express C18, 100 mm×46 mm 5 μm. UV-detection: 214; 254; 280; 320 nm. Eluent A: water with 1%0 formic acid, Eluent B: CH₃CN with 1% formic acid. 0-1 min: 30% B; 1-6 min: 30-100% B; 6-26 min: 100% B; 26-27 min: 100-30% B; 27-30 min: 30% B.
Method 3: Supelco analytical column Ascentis Express C18, 100 mm×46 mm 5 μm. UV-detection: 214; 254; 280; 320 nm. Eluent A: water with 1%0 formic acid, Eluent B: CH₃CN with 1% formic acid. 0-1 min: 30% B; 1-6 min: 30-100% B; 6-8.5 min: 100% B; 8.5-9 min: 100-30% B; 9-16 min: 30% B.
Method 4: Supelco analytical column Ascentis Express C18, 100 mm×46 mm 5 μm. UV-detection: 214; 254; 280; 320 nm. Eluent A: water with 1%0 formic acid, Eluent B: CH₃CN with 1% formic acid. 0-1 min: 30% B; 1-6 min: 30-100% B; 6-8.5 min: 100% B; 8.5-9 min: 100-30% B.
Method 5: Supelco analytical column Ascentis Express C18, 100 mm×46 mm 5 μm. UV-detection: 214; 254; 280; 320 nm. Eluent A: water with 1%0 formic acid, Eluent B: CH₃CN with 1% formic acid. 0-1 min: 30% B; 1-6 min: 30-100% B; 6-8.5 min: 100% B; 8.5-9 min: 100-30% B; 9-13 min: 30% B.
Method 6: Waters Alliance 2695, Supelco Ascentis Express C18, 100 mm×46 mm 5 μm. UV-detection: 214; 254; 280; 320 nm. Eluent A: water with 1%0 formic acid, Eluent B: CH₃CN with 1%0 formic acid. 0-10: 10% B; 10-18 min: 10-95% B; 18-20 min: 95% B; 20-24 min 95-10% B; 24-25 min: 10% B.

### General Procedure for the Preparation of Ureas According to the Method A.

To a solution of the corresponding 2-aminobenzazole (1.0 eq.) in DMF (5 mL/100 mg) at r.t. were added successively sodium hydride (60% in oil, 1.5 eq.), then 20 min later the corresponding isocyanate (1.0 eq.). The resulting solution was stirred at 90° C. until completion of the reaction (overnight, about 18h). After cooling to r.t., the mixture was diluted with ethyl acetate (20 mL/100 mg) and cautiously quenched with water (20 mL/100 mg). The reaction mixture was transferred into a separation funnel and extracted with ethyl acetate. The combined organic layers were washed with water, then with brine, dried with MgSO₄, filtered and concentrated under reduced pressure. The residues were purified by recrystallization from ethanol, or by silicagel flash chromatography to lead to the expected ureas.

General procedure for the preparation of ureas according to the method B.

To a solution of the corresponding aniline (1.0 eq.) in DMF (8 mL/mmol) was added the corresponding isocyanate (1.0 eq.) and the mixture was stirred overnight at rt. After completion of the reaction, the reaction mixture was poured into water (60 mL/mmol) and the precipitate was collected and washed with methanol (2×8 mL/mmol) and diethyl ether (2×8 mL/mmol).

### 2. Compounds

### Example #1: 1-(3-chlorophenyl)-3-(6-nitrobenzo[d]thiazol-2-yl)urea

Compound #1 was synthesized following the general procedure (B) using 2-amino-6-nitrobenzothiazole (500 mg, 2.56 mmol) and 3-chlorophenyl isocyanate (0.28 mL, 2.30 mmol). White powder (802 mg, 90%). ¹H NMR (400 MHz, DMSO-d6): δ 11.44 (s, 1H, N—H), 9.41 (s, 1H, N—H), 8.95 (d, J=1.9 Hz, 1H, H_{Ar}), 8.23 (dd, J=8.9, 2.4 Hz, 1H, H_{Ar}), 7.76 (d, J=8.8 Hz, 1H, H_{Ar}), 7.72 (s, 1H, H_{Ar}), 7.41-7.32 (m, 2H, H_{Ar}), 7.12 (dd, J=8.9, 1.7 Hz, 1H, H_{Ar}); ¹³C NMR (101 MHz, DMSO-d6): δ 164.86, 153.40, 152.37, 142.54, 139.76, 133.29, 131.87, 130.57, 122.93, 121.85, 119.20, 118.73, 118.42, 117.53; HRMS-ESI (m/z): [M+H]⁺ calc. for C₁₄H₁₀ClN₄O₃S⁺, 349.01567; Found: 349.01569. HPLC (λ₂₈₀): Purity 100.0%; t_{R}: 7.708 min (method 5).

### Example #2: 1-(2-chlorophenyl)-3-(6-nitrobenzo[d]thiazol-2-yl)urea

Compound #2 was synthesized following the general procedure (B) using 2-amino-6-nitrobenzothiazole (500 mg, 2.56 mmol) and 2-chlorophenyl isocyanate (0.280 mL, 2.30 mmol) to afford the title compound as a white powder (810 mg, 91%). ¹H NMR (400 MHz, DMSO-d6): δ 11.79 (s, 1H, N—H), 8.93 (s, 1H, N—H), 8.90 (d, J=2.4 Hz, 1H, H_{Ar}), 8.17 (dd, J=8.9, 2.4 Hz, 1H, H_{Ar}), 8.12 (dd, J=8.3, 1.2 Hz, 1H, H_{Ar}), 7.74 (d, J=8.9 Hz, 1H, H_{Ar}), 7.47 (dd, J=8.0, 1.3 Hz, 1H, H_{Ar}), 7.36-7.30 (m, 1H, H_{Ar}), 7.10 (td, J=7.9, 1.4 Hz, 1H, H_{Ar}); ¹³C NMR (101 MHz, DMSO-d6): δ 164.50, 153.86, 151.28, 142.48, 134.53, 132.19, 129.37, 127.74, 124.73, 122.84, 121.66, 121.63, 119.80, 118.58; HRMS-ESI (m/z): [M+H]⁺ calc. for C₁₄H₁₀ClN₄O₃S⁺, 349.01567; Found: 349.01569. HPLC (λ₂₅₄): Purity 97.7%; t_{R}: 10.642 min (method 3).

### Example #3: 1-(6-ethoxybenzo[d]thiazol-2-yl)-3-(o-tolyl)urea

Compound #3 was synthesized following the general procedure (B) using 2-amino-6-ethoxybenzothiazole (500 mg, 2.57 mmol) and 2-tolylisocyanate (0.319 mL, 2.57 mmol). White solid. Yield: 774 mg, 92%. ¹H NMR (200 MHz, DMSO-d6): δ 10.97 (br. s, 1H, N—H), 8.64 (s, 1H, N—H), 7.86 (d, J=7.7 Hz, 1H, H_{Ar}), 7.63 7.42 (m, 2H, H_{Ar}), 7.20 (t, J=8.5 Hz, 2H, H_{Ar}), 7.09 6.89 (m, 2H, H_{Ar}), 4.05 (dd, J=13.7, 6.6 Hz, 2H, CH₂), 2.28 (s, 3H, CH₃), 1.34 (t, J=6.8 Hz, 3H, CH₃); ¹³C NMR (50 MHz, DMSO-d6): δ 157.52, 154.98, 151.65, 143.04, 136.36, 132.58, 130.37, 127.97, 126.36, 123.64, 121.13, 120.41, 114.76, 105.54, 63.58, 17.81, 14.74; HRMS-ESI (m/z): [M+Fi]′ calc. for C₁₇H₁₈N₃O₂S⁺, 328.11142; Found: 328.11154; HPLC (λ₂₈₀): t_{R}: 10.567 min: Purity 97.0% (method 3).

### Example #4: 1-(2-chlorophenyl)-3-(6-methylbenzo[d]thiazol-2-yl)urea

Compound #4 was synthesized following the general procedure (B) using 2-amino-6-methylbenzothiazole (500 mg, 3.05 mmol) and 2-chlorophenylisocyanate (0.368 mL, 3.05 mmol). White solid. Yield: 870 mg, 90%. ¹H NMR (200 MHz, DMSO-d6): δ 11.39 (s, 1H, N—H), 9.14 (s, 1H, N—H), 8.18 (dd, J=8.3, 1.4 Hz, 1H, H_{Ar}), 7.73 (s, 1H, H_{Ar}), 7.63 7.45 (m, 2H, H_{Ar}), 7.42 7.30 (m, 1H, H_{Ar}), 7.22 (dd, J=8.3, 1.2 Hz, 1H, H_{Ar}), 7.12 (td, J=7.6, 1.5 Hz, 1H, H_{Ar}), 2.40 (s, 3H, CH₃); ¹³C NMR (50 MHz, DMSO-d6): δ 158.38, 151.37, 146.74, 134.91, 132.41, 131.37, 129.27, 127.64, 127.18, 124.24, 122.52, 121.48, 121.12, 119.52, 20.83; HRMS-ESI (m/z): [M+H]⁺ calc. for C₁₅H₁₃ClN₃OS⁺, 318.04624; Found: 318.04630; HPLC (λ₂₈₀): t_{R}: 11.367 min: Purity 99.2% (method 3).

### Example #5: 1-(1H-benzo[d]imidazol-2-yl)-3-phenylurea

Compound #5 was synthesized following the general procedure (A) using 2-aminobenzimidazole (133 mg, 1 mmol) and phenylisocyanate (119 mg, 1 mmol), and purified by silicagel flash chromatography (ethyl acetate/cyclohexane, 9/1 to 4/6, v/v). Beige solid. Yield: 25.2 mg, 10%. Rf (Cyclohexane/EtOAc, 75/25, v/v)=0.47; ¹H NMR (200 MHz, DMSO-d6): δ 11.16 (br. s, 2H, 2N—H), 9.57 (s, 1H, N—H), 7.57 (d, J=7.1 Hz, 2H, H_{Ar}), 7.48 7.21 (m, 4H, H_{Ar}), 7.17 6.90 (m, 3H, H_{Ar}); ¹³C NMR (50 MHz, DMSO-d6): δ 154.04, 148.95, 139.36, 135.03 (2C), 128.83 (2C), 122.32, 120.95 (2C), 118.55 (2C), 112.88 (2C); ESI (m/z): [M+H]⁺ calc. for C₁₄H₁₃N₄O⁺, 253.11, found 253.13; HPLC (λ₂₈₀): t_{R}: 5.1 min; Purity 96.4% (method 1).

### Example #6: 1-(1H-benzo[d]imidazol-2-yl)-3-(4-chlorophenyl)urea

Compound #6 was synthesized following the general procedure (A) using 2-aminobenzimidazole (133 mg, 1 mmol) and 4-chlorophenylisocyanate (154 mg, 1 mmol), and purified by silicagel flash chromatography (ethyl acetate/cyclohexane, 9/1 to 4/6, v/v). Pink solid. Yield: 56.0 mg, 19%. Rf (Cyclohexane/EtOAc, 75/25, v/v)=0.52; ¹H NMR (200 MHz, DMSO-d6): δ 11.33 (s, 2H, 2N—H), 9.61 (s, 1H, N—H), 7.63 (d, J=8.6 Hz, 2H, H_{Ar}), 7.34 (d, J=6.1 Hz, 4H, H_{Ar}), 7.07 (dd, J=4.6, 3.4 Hz, 2H, H_{Ar}); ¹³C NMR (50 MHz, DMSO-d6): δ 149.68, 138.79, 138.62, 133.86, 128.58 (2C), 125.51, 121.12 (2C), 119.95 (2C), 119.75, 112.45 (2C); ESI (m/z): [M+H]⁺ calc. for C₁₄H₁₂ClN₄O⁺, 287.07, found 287.13; HPLC (λ₂₈₀): t_{R}: 8.9 min: Purity 96.0% (method 1).

### Example #7: 1-(benzo[d]thiazol-2-yl)-3-(2-chlorophenyl)urea

Compound #7 was synthesized following the general procedure (B) using 2-aminobenzothiazole (500 mg, 3.33 mmol) and 2-chlorophenylisocyanate (0.390 mL, 3.33 mmol). White solid. Yield: 919 mg, 92%. ¹H NMR (200 MHz, DMSO-d6): δ 11.46 (br. s, 1H, N—H), 9.14 (br. s, 1H, N—H), 8.18 (d, J=8.1 Hz, 1H, H_{Ar}), 7.94 (d, J=7.2 Hz, 1H, H_{Ar}), 7.69 (d, J=8.1 Hz, 1H, H_{Ar}), 7.52 (d, J=7.4 Hz, 1H, H_{Ar}), 7.46-7.21 (m, 3H, H_{Ar}), 7.12 (t, J=7.2 Hz, 1H, H_{Ar}); ¹³C NMR (50 MHz, DMSO-d6): δ 159.31, 151.50, 148.93, 134.93, 131.33, 129.39, 127.76, 126.03, 124.48, 123.10, 122.74, 121.71, 121.55, 119.95; HRMS-ESI (m/z): [M+H]⁺ calc. for C₁₄H₁₁ClN₃OS⁺, 304.03059; Found: 304.03064; HPLC (λ₂₈₀): t_{R}: 10.583 min: Purity 95.2% (method 3).

### Example #8: 1-(benzo[d]thiazol-2-yl)-3-(3-chlorophenyl)urea

Compound #8 was synthesized following the general procedure (B) using 2-aminobenzothiazole (500 mg, 3.33 mmol) and 3-chlorophenylisocyanate (0.410 mL, 3.33 mmol). White solid. Yield: 930 mg, 92%. ¹H NMR (200 MHz, DMSO-d6): δ 11.17 (br. s, 1H, N—H), 9.41 (s, 1H, N—H), 7.90 (d, J=7.3 Hz, 1H, H_{Ar}), 7.76 (d, J=1.9 Hz, 1H, H_{Ar}), 7.63 (d, J=7.8 Hz, 1H, H_{Ar}), 7.46-7.30 (m, 3H, H_{Ar}), 7.25 (td, J=7.7, 1.2 Hz, 1H, H_{Ar}), 7.10 (dt, J=7.1, 1.9 Hz, 1H, H_{Ar}); ¹³C NMR (50 MHz, DMSO-d6): δ 160.32, 153.03, 146.80, 140.27, 133.36, 130.70, 130.44, 126.05, 122.95, 122.50, 121.62, 118.71, 118.25, 117.28; HRMS-ESI (m/z): [M+H]⁺ calc. for C₁₄H₁₁C₁N₃OS⁺, 304.03059; Found: 304.0306; HPLC (λ₂₈₀): t_{R}: 10.350 min: Purity 99.4% (method 3).

### Example #9: 1-(benzo[d]thiazol-2-yl)-3-(4-chlorophenyl)urea

Compound #9 was synthesized following the general procedure (B) using 2-aminobenzothiazole (500 mg, 3.33 mmol) and 4-chlorophenylisocyanate (511 mg, 3.33 mmol). White solid. Yield: 889 mg, 88%. ¹H NMR (400 MHz, DMSO-d6): δ 10.97 (br. s, 1H, N—H), 9.33 (s, 1H, N—H), 7.90 (d, J=7.8 Hz, 1H, H_{Ar}), 7.64 (d, J=7.7 Hz, 1H, H_{Ar}), 7.57 (d, J=8.7 Hz, 2H, H_{Ar}), 7.43-7.34 (m, 3H, H_{Ar}), 7.28-7.21 (m, 1H, H_{Ar}); ¹³C NMR (50 MHz, DMSO-d6): δ 160.11, 152.74, 147.16, 137.66, 130.83, 128.77, 126.54, 126.04, 122.95, 121.60, 120.39, 118.95; HRMS-ESI (m/z): [M+H]⁺ calc. for C₁₄H₁₁ClN₃OS⁺, 304.03059; Found: 304.03076; HPLC (λ₂₈₀): t_{R}: 6.917 min: Purity 100.0% (method 4).

Example #10: 1-(benzo[d]thiazol-2-yl)-3-(4-methoxyphenyl)urea Compound #10 was synthesized following the general procedure (B) using 2-aminobenzothiazole (500 mg, 3.33 mmol) and 4-methoxyphenylisocyanate (496 mg, 3.33 mmol). White solid. Yield: 897 mg, 90%. ¹H NMR (200 MHz, DMSO-d6): δ 10.81 (s, 1H, N—H), 9.00 (s, 1H, N—H), 7.90 (d, J=7.8 Hz, 1H, H_{Ar}), 7.65 (d, J=7.9 Hz, 1H, H_{Ar}), 7.50-7.33 (m, 3H, H_{Ar}), 7.23 (t, J=7.5 Hz, 1H, H_{Ar}), 6.92 (d, J=8.9 Hz, 2H, H_{Ar}), 3.73 (s, 3H, OCH₃); ¹³C NMR (50 MHz, DMSO-d6): δ 159.85, 155.32, 152.26, 148.23, 131.45, 131.28, 125.95, 122.83, 121.49, 120.83 (2C), 119.37, 114.13 (2C), 55.19; HRMS-ESI (m/z): [M+H]⁺ calc. for C₁₅H₁₄N₃O₂S⁺, 300.08012; Found: 300.08023; HPLC (λ₂₈₀): t_{R}: 10.092 min: Purity 97.3% (method 2).

### Example #11: 1-(3,5-dichlorophenyl)-3-(6-nitrobenzo[d]thiazol-2-yl)urea

Compound #11 was synthesized following the general procedure (B) using 6-nitro-2-aminobenzothiazol (4.00 g, 26.00 mmol) and 1,3-dichloro-5-isocyanatobenzene (4.50 g, 24.00 mmol). Yellow powder. Yield: 6.44 g, 70%. ¹H NMR (400 MHz, Acetone-d6): δ 11.20 (br.s, 1H), 9.65 (br. s, 1H), 9.34 (s, 1H), 8.74 (d, J=7.1 Hz, 1H), 8.43 (s, 1H), 8.25 (d, J=8.9 Hz, 1H), 7.63 (s, 1H); ¹³C NMR (101 MHz, Acetone-d6): 165.6, 162.9, 144.5, 141.8, 135.8, 133.2, 123.7 (2C), 122.7, 120.4, 119.1, 118.4 (2C), 118.3; HRMS-ESI (m/z): [M+H]⁺ calcd for C₁₄H₇Cl₂N₄O₃S, 382.97665 Found: 382.97669; HPLC (λ₂₅₄): Purity 98.0%; t_{R}: 3.78 min (method 6).

### Example #12: 1-(4-bromophenyl)-3-(6-nitrobenzo[d]thiazol-2-yl)urea

Compound #12 was synthesized following the general procedure (B) using using 6-nitro-2-aminobenzothiazol (2.00 g, 13.30 mmol) and 1-bromo-4-isocyanatobenzene (2.39 g, 12.09 mmol). White powder. Yield 3.77 g, 79%. ¹H NMR (200 MHz, DMSO-d6): δ 11.33 (br. s, 1H), 9.37 (s, 1H), 8.99 (s, 1H), 8.25 (dd, J=8.9, 2.3 Hz, 1H), 7.79 (d, J=8.3 Hz, 1H), 7.52 (s, 4H), 7.43 (s, 1H); ¹³C NMR (101 MHz, DMSO-d6) δ 164.85, 152.25, 142.52, 138.95, 137.61, 132.05, 131.71, 131.51, 121.83, 120.98, 120.23, 118.71, 114.89, 113.38; HRMS-ESI (m/z): [M+H]⁺ calcd for C₁₄H₁₀BrN₄O₃S, 392.9652; Found: 392.9652; HPLC (λ₂₅₄): Purity>99.9%; t_{R}: 12.60 min (method 6).

### Example #13: 1-(2-bromophenyl)-3-(6-nitrobenzo[d]thiazol-2-yl)urea

Compound #13 was synthesized following the general procedure (B) using 6-nitro-2-aminobenzothiazol (420.50 mg, 2.80 mmol) and 1-bromo-2-isocyanatobenzene (500.00 mg, 2.50 mmol). White powder. Yield 655.30 mg, 66%. ¹H NMR (200 MHz, DMSO-d6): δ 11.92 (br. s, 1H), 8.98 (d, J=1.9 Hz, 1H), 8.86 (s, 1H), 8.23 (dd, J=8.9, 2.0 Hz, 1H), 8.05 (d, J=7.9 Hz, 1H), 7.81 (d, J=8.9 Hz, 1H), 7.67 (d, J=7.9 Hz, 1H), 7.40 (t, J=7.7 Hz, 1H), 7.08 (t, J=7.5 Hz, 1H); ¹³C NMR (101 MHz, DMSO-d6): 164.71, 162.38, 154.04, 151.50, 142.62, 135.68, 132.76, 132.25, 128.36, 125.72, 122.92, 121.81, 119.99, 118.76, 114.16; HRMS-ESI (m/z): [M+H]⁺ calc for C₁₄H₁₀BrN₄O₃S, 392.9649; Found: 392.9651 HPLC (λ₂₅₄): Purity 96.3%; t_{R}: 12.23 min (method 6).

### Example #14: 1-(3-chlorophenyl)-3-(6-nitro-1H-benzo[d]imidazol-2-yl)urea

Compound #14 was synthesized following the general procedure (B) using 6-nitro-1H-benzo[d]imidazol-2-amine (500 mg, 2.80 mmol) and 3-chlorophenyl isocyanate (0.34 mL, 2.80 mmol). White powder. Yield: 667 mg, 72%). ¹H NMR (200 MHz, DMSO-d6): δ 11.65 (br. s, 2H), 9.81 (s, 1H), 8.26 (d, J=2.0 Hz, 1H), 8.03 (dd, J=8.8, 2.2 Hz, 1H), 7.82 (s, 1H), 7.53 (d, J=8.8 Hz, 1H), 7.44-7.30 (m, 2H), 7.16-7.05 (m, 1H). ¹³C NMR (50 MHz, DMSO-d6): δ 152.53, 151.50, 142.40, 141.58, 140.34, 135.38, 133.35, 130.54, 122.46, 118.14, 117.36, 117.12, 113.52, 109.07. HRMS-ESI (m/z): [M+H]⁺ calc. for C₁₄H₁₁ClN₅O₃⁺, 332.05449; Found: 332.05447; HPLC (λ₂₈₀): Purity 99.4%; t_{R}: 19.183 min (method 1).

### Comparative example #5-1: 1-(benzo[d]oxazol-2-yl)-3-phenylurea

Compound #5-1 was synthesized following the general procedure (A) using 2-aminobenzoxazole (134 mg, 1 mmol) and phenylisocyanate (119 mg, 1 mmol), and purified by recrystallization from ethanol. Brown solid. Yield: 59.3 mg, 24%. Rf (Cyclohexane/EtOAc, 70/30, v/v)=0.48; ¹H NMR (200 MHz, DMSO-d6): δ (ppm): 6.96 (t, J=7 Hz, 1H, H_{Ar}), 7.27 (t, J=7 Hz, 2H, H_{Ar}), 7.35-7.55 (m, 6H, H_{Ar}), 8.73 (s, 1H, N—H); ¹³C NMR (50 MHz, DMSO-d6): δ (ppm): 118.5 (2C), 122.0, 129.0 (2C), 129.1, 129.1 (2C), 129.2 (2C), 135.0, 140.0, 149.2, 152.9; ESI (m/z): [M+H—NHCO]⁺ calc. for C₁₃H₁₁N₂O⁺, 211.09, found 211.27; HPLC (λ₂₈₀), t_{R}: 16.8 min: Purity 98.3% (method 1).

### Example B: Biology

### I. Material and Methods

### Reagents and Antibodies

Sunitinib, SB203580, SB225002, cisplatine and Danirixin were purchased from Selleckchem (Houston, USA). Anti-HSP60 antibodies were purchased from Santa Cruz Biotechnology (Santa Cruz, Calif., USA). Anti-AKT anti-phospho-AKT, anti-ERK, anti-phospho-ERK antibodies were from Cell Signaling Technology (Beverly, Mass., USA).

### Cell Culture

RCC4, 786-0 (786) and A498 (498) RCC cell lines, MDA-MD-231 breast cell line, Cal27 and Cal 33 head and neck cell line were purchased from the American Tissue Culture Collection (ATTC). Resistant cells 786R (resistant to Sunitinib), CAL27RR (resistant to multi-irradiations by photons and cisplatin), and CAL33RR (resistant to multi-irradiations by photon and cisplatin) were provided by the inventors. OCI-AML2, OCI-AML3, Molm13 and Molm14 acute myeloid cell lines (AML), and K562 chronic myeloid cell line (CML), SKM1 myelodysplastic cell line (MDS) were provided by Dr. P. Auberger (C3M, Nice, France). Primary cells (CC, TF and 15S) were already described and cultured in a medium-specific for renal cells (PromoCell, Heidelberg Germany).

### Immunoblotting

Cells were lysed in buffer containing 3% SDS, 10% glycerol and 0.825 mM Na₂HPO₄. 30 to 50 μg of proteins were separated on 10% SDS-PAGE, transferred onto a PVDF membrane (Immobilon, Millipore, France) and then exposed to the appropriate antibodies. Proteins were visualized with the ECL system using horseradish peroxidase-conjugated anti-rabbit or anti-mouse secondary antibodies.

### Migration Assay

CXCL7 or VEGFA-stimulated chemotaxis assays were monitored using modified Boyden chambers containing polycarbonate membranes (8-μm pores, Transwell; Corning, Sigma). Cells were seeded on the upper side of the filters and chambers were placed on 24-well plates containing CXCL7 (50 ng/ml) or VEGFA (50 ng/ml). Cells were allowed to migrate for 24 hr at 37° C. in 5% CO₂. Migratory cells on the lower membrane surface were fixed in 3% paraformaldehyde, stained with 0.1% crystal violet.

### Colony Formation Assay

Cells (5000 cells per condition) were treated or not with compound #1 and sunitinib, and cisplatin. Colonies were detected after 10 days of culture. Cells were then washed, fixed at room temperature for 20 min with 3% paraformaldehyde (PFA; Electron Microscopy Sciences) and colored by GIEMSA (Sigma)

### Caspase Assays

Caspase 3 activity was assessed in quadruplicates using z-DEVD-AMC as substrate and fluorescence were assessed.

### Flow Cytometry

CXCR2 measurement: After stimulation, cells were washed with PBS and were stained with the CXCR2-PE antibody (Miltenyi) for 30 min at room temperature. Fluorescence was measured by using the FL2 (PE) channels of a fluorescence-activated cell sorter apparatus (Calibur cytometer).

Apoptosis analysis: After stimulation, cells were washed with ice-cold PBS and were stained with the annexin-V-fluos staining kit (Roche, Meylan, France) according to the manufacturer's procedure. Fluorescence was measured by using the FL2 (AV) and FL3 (propidium iodide, PI) channels of a fluorescence-activated cell sorter apparatus (Calibur cytometer).

Cell viability (XTT)

Cells (5×10³ cells/100 μl) were incubated in a 96-well plate with different effectors for the times indicated in the figure legends. Fifty microliters of sodium 3′-[1-phenylaminocarbonyl)-3,4-tetrazolium]-bis(4-methoxy-6-nitro) benzene sulfonic acid hydrate (XTT) reagent was added to each well. The assay is based on the cleavage of the yellow tetrazolium salt XTT to form an orange formazan dye by metabolically active cells. The absorbance of the formazan product, reflecting cell viability, was measured at 490 nm. Each assay was performed in quadruplicate.

Quantitative Real-Time PCR (qPCR) Experiments

One microgram of total RNA was used for the reverse transcription, using the QuantiTect Reverse Transcription kit (QIAGEN, Hilden, Germany), with a blend of oligo (dT) and random primers to prime first-strand synthesis. SYBR master mix plus (Eurogentec, Liege, Belgium) was used for qPCR. The mRNA level was normalized to 36B4 mRNA.

### Tumor Xenograft Experiment

Ectopic model of RCC: seven million A498 cells were injected subcutaneously into the flank of 5-week-old nude (nu/nu) female mice (Janvier, France). The tumor volume was determined with a caliper (v=L*1²*0.5). When the tumor reached 100 mm³, mice were treated five a week for 4 weeks, by gavage with placebo (dextrose water vehicle) or compound #1 (50 mg/kg). This study was carried out in strict accordance with the recommendations in the Guide for the Care and Use of Laboratory Animals. Our experiments were approved by the “Comite national institutionnel d′éthique pour l'animal de laboratoire (CIEPAL)”

### Immunohistochemistry

Sections from blocks of formol-fixed and paraffin-embedded tumors were examined for immunostaining. Sections were incubated with monoclonal anti-Ki67 (clone MIB1, DAKO, Ready to use) antibodies. Biotinylated secondary antibody (DAKO) was applied and binding was detected with the substrate diaminobenzidine against a hematoxylin counterstain.

### Gene Expression Microarray Analysis

Normalized RNA sequencing (RNA-Seq) data produced by The Cancer Genome Atlas (TCGA) were downloaded from cBiopotal (www.cbioportal.org, TCGA Provisional; RNA-Seq V2).

### Statistical Analysis

All data are expressed as the mean±the standard error (SEM). Statistical significance and p values were determined by the two-tailed Student's t-test. One-way ANOVA was used for statistical comparisons. Data were analyzed with Prism 5.0b (GraphPad Software) by one-way ANOVA with Bonferroni post hoc.

### 2. Results

### 2.1 In Vitro Tests

Compounds #1-#14 have been assayed as potential anti-proliferative agents against a panel of human breast head and neck, and kidney tumor cells and hematological malignancies, selected on the basis of their aggressiveness (e.g. incurable triple-negative breast cancer cells: MDA-MB-231; kidney ccRCC cells: RCC4, A498 and 786-O and 786R; head and neck cancer cells: CAL33, CAL33RR, CAL27, and CAL27RR; acute myeloid cell lines: OCI-AML2, OCI-AML3, MolM13, and MolM14; myelodysplastic cell line: SM1; and chronic myeloid cell line: CML). All these cell lines share as common denominator the expression of CXCR1 and CXCR2. The EC₅₀ values have been measured by a XTT colorimetric assay, and compared to those of SB-225002, used as a reference; the results are listed in Table 1.

TABLE 1
Evaluation of compounds #1-#10 against different solid hematological tumor cell lines.

| | Breast | Kidney | | | | Head and Neck | | | |
|---|---|---|---|---|---|---|---|---|---|
| EC₅₀ (μM) | MDA-MB-231 | A498 | RCC4 | 786-O | 786R | CAL33 | CAL33RR | CAL27 | CAL27RR |
| #1 | 2.5 | 2.5 | 5 | 2 | 2.5 | 2 | 3 | 3 | 2 |
| #2 | 8 | 5 | 7 | 5 | 8 | | | | |
| #3 | 12 | 15 | >25 | 15 | | | | | |
| #4 | >25 | 15 | >25 | 15 | | | | | |
| #5 | 12 | 10 | 25 | 20 | | | | | |
| #6 | 20 | 9 | 20 | 20 | | | | | |
| #7 | >25 | >25 | 25 | >25 | | | | | |
| #8 | >25 | >25 | 20 | >25 | | | | | |
| #9 | >25 | 25 | 20 | >25 | | | | | |
| #10 | >25 | >25 | 15 | >25 | | | | | |
| #11 | | 2.5 | 2 | 5 | 5 | 4 | 4 | 2.5 | 2.5 |
| #12 | | 7 | 7 | 5 | 5 | <2.5 | 20 | 5 | 2.5 |
| #13 | | 20 | 25 | 15 | 14 | 8 | >25 | >25 | |
| #14 | | 5 | 3 | 4 | 4 | 15 | 25 | 15 | 10 |
| Comp. | >25 | >25 | >25 | >25 | | | | | |
| #5-1 | | | | | | | | | |
| Comp. | 100 | 70 | 100 | 100 | | | | | |
| SB-225002 | | | | | | | | | |

| | AML | | | | MDS | CML |
|---|---|---|---|---|---|---|
| EC₅₀ (μM) | OCI-AML2 | OCI-AML3 | MolM13 | MolM14 | SKM1 | K562 |
| #1 | 5 | 4 | 7 | 4 | 5 | 2 |
| #2 | 10 | 10 | 5 | 10 | 10 | 7 |
| #3 | 15 | 7 | 15 | 10 | 15 | 4 |
| #4 | >25 | 10 | 15 | 10 | 25 | 25 |
| #5 | 18 | 25 | 25 | 20 | 25 | 10 |
| #6 | 20 | 22 | >25 | 20 | >25 | 12 |
| #7 | 10 | >25 | >25 | >25 | >25 | >25 |
| #8 | >25 | >25 | >25 | >25 | >25 | 25 |
| #9 | >25 | >25 | >25 | >25 | >25 | 25 |
| #10 | >25 | >25 | >25 | >25 | >25 | 25 |
| #11 | | | | | | |
| #12 | | | | | | |
| #13 | | | | | | |
| #14 | | | | | | |
| Comp. | >25 | >25 | >25 | >25 | >25 | >25 |
| #5-1 | | | | | | |
| Comp. | >100 | 90 | >100 | 90 | >100 | >100 |
| SB-225002 | | | | | | |

Values are reported as IC50 measured by XTT assay (48 h), the results are expressed in μM, and all the IC50 values given in this table show a standard deviation of 10%.

The results show that compounds of the invention exert stronger cytotoxic activities than the reference molecule SB-225002 and 1-(benzo[d]oxazol-2-yl)-3-phenylurea (example #5-1) used also as a comparative example.

The results further show that substituted compounds in R₁ position (examples #1-#4 and #11-14) exert stronger cytotoxic activities than unsubstituted compounds in R₁ position. More particularly, compounds #1, #2, and #11-14 substituted by a nitro group in R₁ position and comprising a chlorophenyl exert the strongest activity.

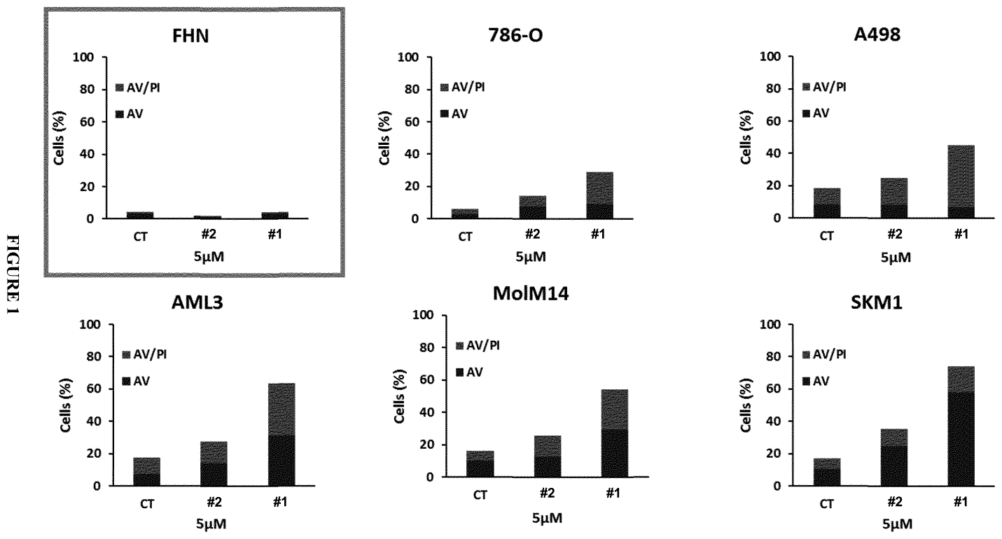

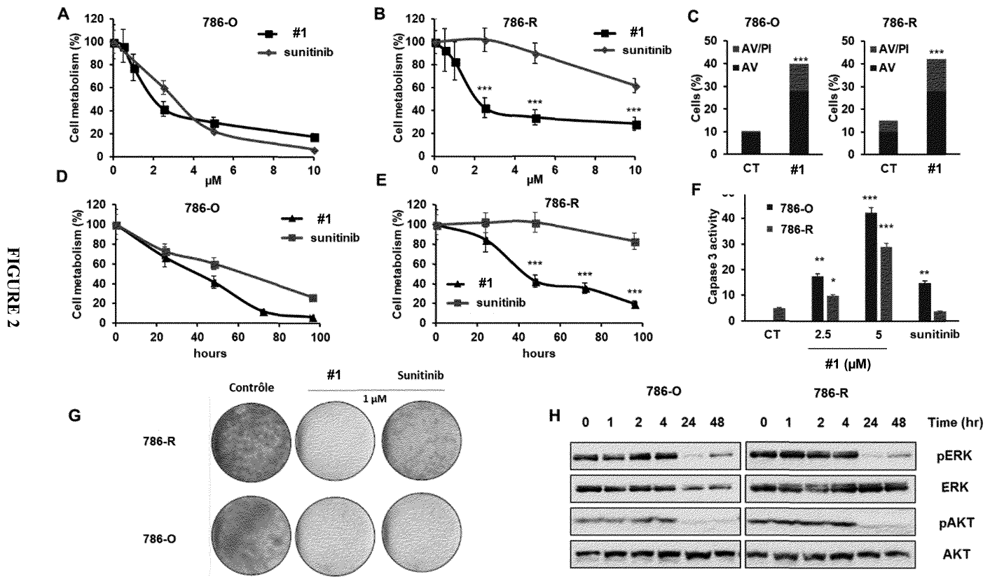

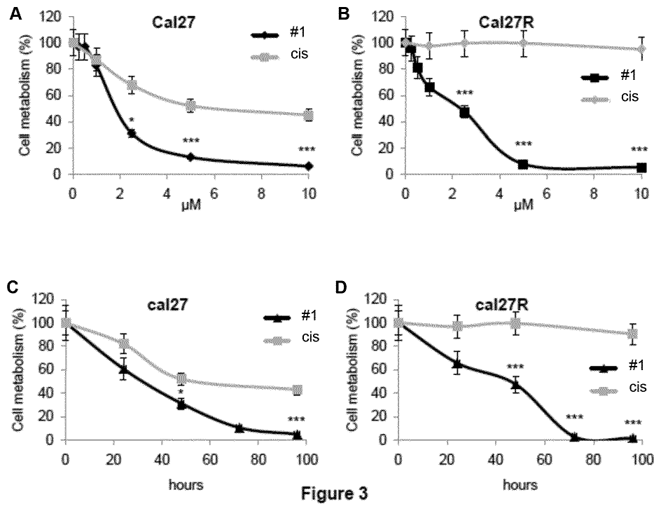

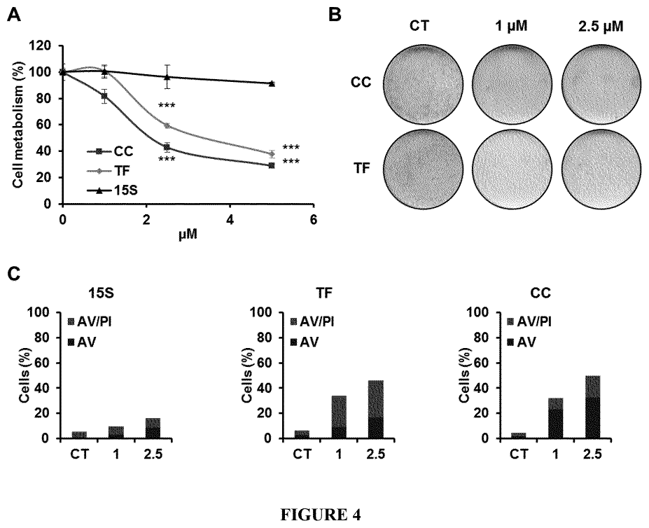

To complete these XTT assays, malignant (ccRCC and hematological) and healthy (human fibroblasts FHN) cells have been grown with compounds #1 or #2, and after 48h the expression of cell death markers (AV and PI) has been quantified by FACS analyses (FIG. 1).

The results show that compounds #1 or #2 exert a cytotoxic effect on malignant cells. Compound #1 appears more efficient than compound #2 in inducing early (AV) and late (PI) apoptosis makers. Some discrepancies are also observed depending on the cell lines. For example, the early apoptosis proportion is much higher in ccRCC (786-O and A498) than in hematological tumor cells (AML, MolM14, and SKM1). FACS analyses of healthy human fibroblasts (FHN) showed no increased apoptosis over the control experiment suggesting that compounds #1 and #2 are not toxic on normal tissues. These results confirm the data obtained in the XTT assays and have allowed to select compound #1 for further biological investigations.

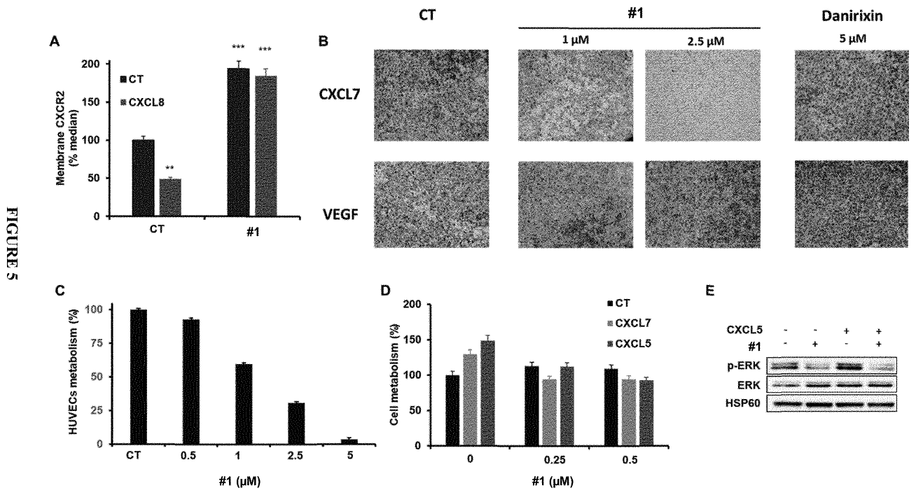

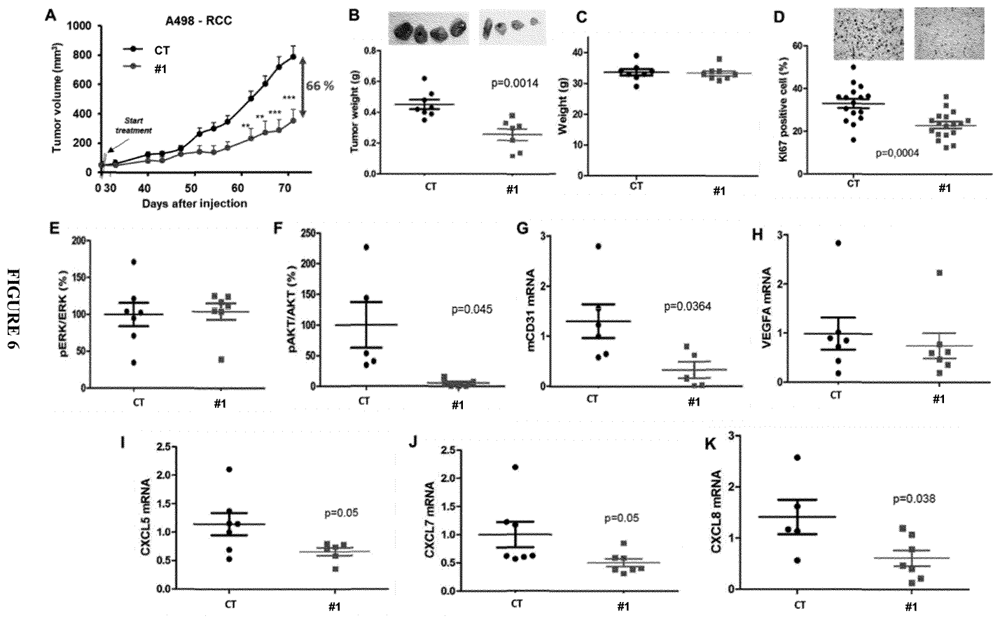

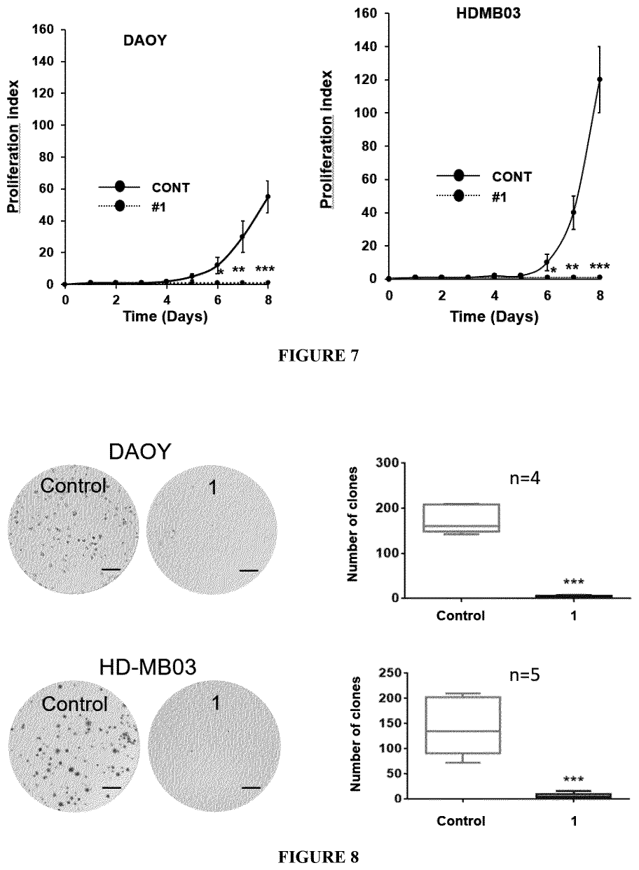

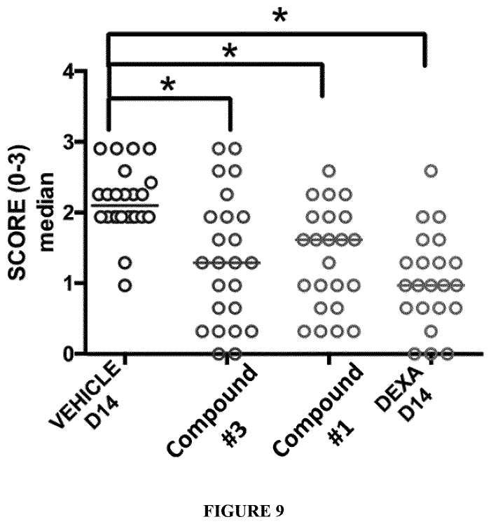

The cytotoxic and cytostatic effects of compound #1 against sunitinib-sensitive and -resistant cells have also been evaluated and results thereof are detailed in FIG. 2.

Compound #1 remarkably retains its cytotoxic effects against the sensitive and resistant 786-O cells, as illustrated by the dose-response curves (FIGS. 2A and 2B) and by the corresponding EC₅₀ values (2 μM in both cases). In addition, the treatment of these two types of malignant cells with compound #1 at 2.5 μM (approximately the EC₅₀ concentration) leads to a total inhibition of their proliferation after approximately 65 hours (786-O cells) and 100 hours (786-R cells) of treatment (FIGS. 2D and 2E).

The FACS analyses of death markers revealed that compound #1 is a cytotoxic agent, which kills in a similar way sensitive and resistant (FIG. 2C) cells. Cell death induction may be related to an increase in caspase-3 activity, which is significantly strengthened by compound #1 in both cell lines when used at 2.5 μM (FIG. 2F). In addition, the clonogenicity assays unambiguously demonstrate that compound #1 also exerts a cytostatic effect against both 786-O and 786-R cells (FIG. 2G).

Lastly, compound #1 inhibits the phosphorylation of ERK and AKT, which are activated through the stimulation of CXCR receptors by CXCL cytokines. Importantly, these kinases are at the crossroad of several cellular signaling pathways leading to proliferation, pro-survival and the pro-angiogenic processes. These results confirm that compound #1 inhibits the ERL+CXCL/CXCR pathway (FIG. 2H).

The cytotoxic effect of compound #1 against cisplatin-sensitive and -resistant cells has also been evaluated and results thereof are detailed in FIG. 3.

Compound #1 remarkably retains its cytotoxic effects against the sensitive and resistant Cal27 cells, as illustrated by the dose-response curves (FIGS. 3A and 3B) and by the corresponding EC₅₀ values (2 μM in both cases). In addition, the treatment of these two types of malignant cells with compound #1 at 2.5 μM (approximately the EC₅₀ concentration) leads to a total inhibition of their proliferation after approximately 70 hours (Cal27 cells) and 70 hours (Cal27R cells) of treatment (FIGS. 3C and 3D).

The biological effects of compound #1 on primary RCC tumors cells and normal renal cells harvested from patients suffering from kidney cancer have been explored (FIG. 4).

It has been observed that compound #1 significantly decreases the proliferation of primary kidney tumor cells (FIG. 4A, EC50 in the 2 μM, TF and CC; and FIG. 4B) but has no effect on primary normal kidney cells (15S), even when compound #1 was used at a higher concentration (5 μM). In addition, FACS analysis puts into exergue the apoptosis markers in TF and CC cells cultured in presence of compound #1 at 1 μM, which is not the case with healthy cells 15S (FIG. 4C).

The potential use of compound #1 as an anti-angiogenic agent has further been evaluated on healthy endothelial cells (HuVECs) (FIG. 5).

Since CXCR2 is internalized in endothelial cells when activated by CXCL-8, the effect of compound #1 has been investigated on CXCR2 recycling on HuVEC cells. Following CXCL-8 stimulation in the presence of compound #1 (2.5 μM), CXCR2 is locked at the membrane surface attesting therefore that compound #1 prevents the CXCL-8-dependent internalization of CXCR2 (FIG. 5A).

In addition, compound #1 decreases by more than 50% HuVECs motility (Boyden chamber assays, FIG. 5B). Conversely, when HuVECs are stimulated by VEGFA, compound #1 does not exert any visible activity at the same concentration, underlying that this molecule specifically inhibits the CXCL7-dependent stimulation of CXCR receptors. Importantly, danirixin, a potent antagonist of the ELR+CXCL/CXCR2 interaction (EC50 in the 15 nM range), which reached phase II clinical trials for the treatment of the Respiratory Syncytial Virus (RSV) infection, appears less efficient than compound #1 in reducing HuVEC CXCL7-dependent motility (FIG. 5B).

Moreover, compound #1 also inhibits basal and CXCL5/CXCL7-dependent HuVEC proliferation (FIGS. 5C and 5D), which is consistent with the inhibition of the ERK signaling pathway (FIG. 5E).

### 2.2 In Vivo Tests

The stability of compound #1 was first assayed through UPLC/HRMS analyses in cellulo on the 786-O cell line. No degradation of the hit was observed after a 24h treatment at room temperature, attesting, therefore, a high stability of compound #1.

Compound #1 was then formulated at 7.6 mg/mL, and administrated by oral gavage at 50 mg/Kg (n=12 mice) and pharmacokinetic parameters have been measured (Table 2).

TABLE 2

| | | |
|---|---|---|
| t_{1/2} | 191 ± 43 | min |
| AUC last_{PO} | 84965 ± 9367 | min · ng/mL |
| Tₘₐₓ | 30 | min |

| | |
|---|---|
| Cₘₐₓ | 2.6 nmol/mL (0.9 μg/mL) |

Compound #1 exhibits a remarkable half-life time over 190 min, combined with a C_{MAX} of 0.9 μg/ml at 30 minutes. The global exposure remains high, and the AUC is close to 85000 min·ng/mL.

Compound #1 has further been evaluated on the growth of tumors in mice (FIG. 6).

Mice were xenografted with the highly aggressive ccRCC cells (A498), which form highly vascularized tumors. Following subcutaneous inoculation of 7.106 cells, tumors of approximately 100 mm³ developed within 30 days, it has been observed that compound #1 prevents significantly tumor growth since, at the end of the experiment (day 70), the tumor volume was reduced by more than 65%. This result may be correlated with the reduction of the tumor weight by more than 35% (FIGS. 6A and 6B). No weight loss of the animals in the treated group has been observed, which suggests that the compound does not exert acute toxicity (FIG. 6C).

Immunostaining assays revealed that compound #1 significantly decreases the labelling of the proliferation marker Ki-67 (FIG. 6D). Analyses of tumor lysates show that compound #1 inhibits AKT but not ERK phosphorylation (FIGS. 6E and 6F). The mRNA levels of murine CD31 (FIG. 6G), a relevant marker of blood vessels, are lowered by more than 75% in the group of treated animals. The mRNA levels of ERL+CXCL cytokines (CXCL5 (FIG. 6I), CXCL7 (FIG. 6J), and CXCL8 (FIG. 6K)), but not those of VEGFA (FIG. 6H), are significantly decreased by compound #1, which is consistent with a down-regulation of CD31 levels.

### Example C: Medulloblastoma

### I. Material and Methods

### Cell Culture

DAOY and HD-MBO3 cell lines were purchased from the ATCC. They were cultivated at 37° C. in an incubator with a MEMα (1X)+Glutamax (Invitrogen®) in which fetal calf serum 10% (D. Dutscher) and sodium pyruvate 1 mM (Gibco® Life Technologies).

### Cell Viability (XTT)

5000 DAOY cells and 50000 HD-MB03 cells were incubated in a 96-well plate with different inhibitor concentrations for 24h and 48 hours. A control without cell has been performed. Fifty microliters of sodium 3′-[1-phenylaminocarbonyl)-3,4-tetrazolium]-bis(4-methoxy-6-nitro) benzene sulfonic acid hydrate (XTT) reagent was added to each well. The assay is based on the cleavage of the yellow tetrazolium salt XTT to form an orange formazan dye by metabolically active cells. The absorbance of the formazan product, reflecting cell viability, was measured at 450 nm. Each assay was performed in triplicate.

### Proliferation Test

1500 DAOY and HD-MB03 cells were seeded in a 6-well plate with or without treatment (1 μM of compound #1 or DMSO as control “CONT”), at 37° C. in a 5% CO₂ incubator. Cells were dissociated using trypsone 1X-EDTA (Gibco® Life Technologies) and were counted with Coulter Beckman® counter (Villepinte) at days 1, 4, 6, 7, and 8. Each assay was performed in triplicate.

### Clonogenicity

150 DAOY cells and 300 HD-MB03 cells were seeded in a 6-well plate with or without treatment (1 μM of compound #1 or DMSO as control “CONT”). At Day+7 (DAOY) and Day+11 (HD-MB03), cells were mixed with absolute ethanol for 20 minutes, washed with PBS and colored with Giemsa 50% for 30 minutes. After washing, the boxes were scanned and the number of clones was quantified using ImageJ® software. Each assay was performed in duplicate.

### 2. Results

The results of FIG. 7 show that compound #1 significatively reduces the proliferation of DAOY and HD-MB03 cells compared to the control cells treated with DMSO.

The results of FIG. 8 show that compound #1 significatively reduces the formation of DAOY and HD-MB03 clones.

### Example D: Macular Degeneration

The compounds of the invention have been evaluated in a macular degeneration model.

### I. Protocol

4 groups of 12 mice were induced on the eyes by laser burnt as follows:
   G1: vehicle;
   G2 and G3: Compounds #1 and #3: intraperitoneal injection of 400 μg of product 3 times a week; and
   G4: dexamethasone (2 mg/kg/day) orally.

### 2. Results

Clinical angiography at Day 14 on the 12 animals: evaluation of the intensity of the lesion by a score of 0 to 3 (0 no leak, 1=light intensity, 2=moderate intensity, 3=intense marking).

A significant effect of the treatment with inhibitors, particularly compound #3, is observed at D14, on angiograms in vivo (FIG. 9).

1-20. (canceled)

21. A method of treating cancer comprising administering to a subject in need of treatment a compound of formula (I), a pharmaceutically acceptable salt, a tautomer, or a pharmaceutical composition thereof: 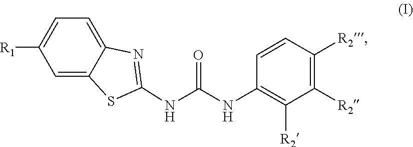
wherein:
R₁ is a radical selected from the group consisting of a nitro group, a (C₁-C₆)alkyl group, and a (C₁-C₆)alkyloxy group;
R_{2′}, R_{2″}, and R_{2″} represent independently a hydrogen, a halogen, or a (C₁-C₆)alkyl group, wherein two substituents chosen among R_{2′}, R_{2″}, and R_{2″} are a hydrogen and the other is a halogen or a (C₁-C₆)alkyl group; and
with the proviso that the compound of formula (I) is not a compound selected from the group consisting of:
1-(4-chlorophenyl)-3-(6-methoxybenzo[d]thiazol-2-yl)urea;
1-(3-fluorophenyl)-3-(6-methoxybenzo[d]thiazol-2-yl)urea;
1-(6-nitrobenzo[d]thiazol-2-yl)-3-o-tolylurea; and
1-(6-nitrobenzo[d]thiazol-2-yl)-3-m-tolylurea.

22. The method according to claim 21, wherein R₁ is a radical selected from the group consisting of a nitro group, a methyl group, and an ethoxy group.

23. The method according to claim 21, wherein R_{2′}, R_{2″}, and R_{2″} represent independently a hydrogen, a chlorine atom, a bromine atom, or a methyl group, wherein two substituents chosen among R_{2′}, R_{2″}, and R_{2″} are a hydrogen and the other is a chorine atom, a bromine atom or a methyl group.

24. The method according to claim 21, wherein:
R₁ is a nitro group; and
R_{2′}, R_{2″}, and R_{2′″} represent independently a hydrogen, a chlorine atom or a bromine atom, wherein two substituents chosen among R_{2′}, R_{2″}, and R_{2″} are a hydrogen and the other is a chlorine atom or a bromine atom.

25. The method according to claim 21, wherein said compound is selected from the group consisting of:
1-(3-chlorophenyl)-3-(6-nitrobenzo[d]thiazol-2-yl)urea;
1-(2-chlorophenyl)-3-(6-nitrobenzo[d]thiazol-2-yl)urea;
1-(6-ethoxybenzo[d]thiazol-2-yl)-3-(o-tolyl)urea;
1-(2-chlorophenyl)-3-(6-methylbenzo[d]thiazol-2-yl)urea;
1-(4-bromophenyl)-3-(6-nitrobenzo[d]thiazol-2-yl)urea; and
1-(2-bromophenyl)-3-(6-nitrobenzo[d]thiazol-2-yl)urea.

26. The method according to claim 21, wherein said cancer is selected from the group consisting of a medulloblastoma, a head and neck cancer, a kidney cancer, and a triple-negative breast cancer.

27. The method according to claim 26, wherein the subject has a head and neck cancer in a subject resistant to cisplatin, oxaliplatin, or carboplatin.

28. The method according to claim 26, wherein the subject has a kidney cancer resistant to sunitinib, axitinib, or cabozantinib.

29. The method according to claim 21, wherein a pharmaceutical composition comprising a compound of formula (I) or a pharmaceutically acceptable salt thereof is administered to the subject.

30. The method according to claim 29, wherein said composition is administered at a dose from 1 to 1000 mg/kg body weight (BW).

31. The method according to claim 29, wherein said composition is administered orally or parenterally.

32. A method of treating a cancer selected from the group consisting of a medulloblastoma, a head and neck cancer and a kidney cancer comprising administering to a subject in need of treatment a compound of formula (II), a pharmaceutically acceptable salt, a tautomer, or a pharmaceutical composition thereof: 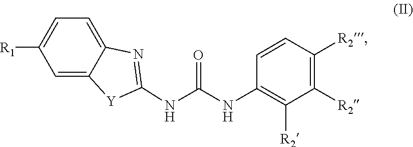
wherein:
Y is NH or S;
R₁ is a radical selected from the group consisting of a hydrogen atom, a nitro group, a (C₁-C₆)alkyl group, and a (C₁-C₆)alkyloxy group;
R_{2′}, R_{2″}, and R_{2″} represent independently a hydrogen atom, a halogen atom, a (C₁-C₆)alkyl group, or a (C₁-C₆)alkyloxy group;
for use for treating.

33. The method according to claim 32, wherein said compound is selected from the group consisting of:
1-(3,5-dichlorophenyl)-3-(6-nitrobenzo[d]thiazol-2-yl)urea; and
1-(3-chlorophenyl)-3-(6-nitro-1H-benzo[d]imidazole-2-yl)urea.

34. A compound, a salt or a tautomer thereof, selected from the group consisting of:
1-(2-chlorophenyl)-3-(6-nitrobenzo[d]thiazol-2-yl)urea;
1-(6-ethoxybenzo[d]thiazol-2-yl)-3-(o-tolyl)urea;
1-(2-chlorophenyl)-3-(6-methylbenzo[d]thiazol-2-yl)urea;
1-(3,5-dichlorophenyl)-3-(6-nitrobenzo[d]thiazol-2-yl)urea;
1-(4-bromophenyl)-3-(6-nitrobenzo[d]thiazol-2-yl)urea;
1-(2-bromophenyl)-3-(6-nitrobenzo[d]thiazol-2-yl)urea; and
1-(3-chlorophenyl)-3-(6-nitro-1H-benzo[d]imidazole-2-yl)urea.

35. A pharmaceutical composition comprising a compound according to claim 34 and a pharmaceutically acceptable carrier.
